(19) United States
(12) Patent Application Publication
(10) Pub. No.: US 2003/0049651 A1
(43) Pub. Date: Mar. 13, 2003

(54) Tumor associated proteins

(75) Inventors: Liu-Ying Luo, Toronto (CA); Eleftherios P. Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto (CA)

(21) Appl. No.: 10/092,012

(22) Filed: Mar. 5, 2002

Related U.S. Application Data
(60) Provisional application No. 60/273,502, filed on Mar. 5, 2001.

(51) Int. Cl. C12Q 1/68; C07H 21/04; C12N 9/00; A61K 39/00; C12P 21/02; C12N 5/06; C07K 16/40

(52) U.S. Cl. 435/6; 435/69.1; 435/183; 435/320.1; 435/325; 530/388.26; 536/23.2

(57) ABSTRACT

Proteins that trigger immune responses in cancer patients are described. The proteins include protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); and, plakophilin 4 (PKP4) (Genbank NM-003628). In addition, a novel nucleic acid molecule identified in an immunoscreen of a breast carcinoma cDNA library is described. Diagnostic and therapeutic applications are also described.

### FIELD OF THE INVENTION

The invention relates to the identification of tumor associated proteins, novel nucleic acid molecules encoding tumor associated proteins and proteins encoded by such nucleic acid molecules; and uses of the tumor associated proteins.

### BACKGROUND OF THE INVENTION

Cancer is one of the leading causes of death in humans. In North America, it is estimated that one in four people will die of cancer. The etiology of cancer is still not clear. Although some cancers have specific etiology, in general, it is thought that both genetic factors and environmental carcinogens contribute to disease pathogenesis.

In cancer, it is now known that certain cellular components may act as immunogens, thus triggering autoimmune responses. The underlying mechanisms include (a) failure of self-tolerance of the immune system; (b) aberrant expression level of certain cellular components; and, (c) expression of mutated forms of certain cellular components. The circulating autoantibodies to such antigens have potential for cancer diagnosis, monitoring or prognosis, and for developing therapeutic cancer vaccines (Zouali, M., Clin Exp Rheumatol. 12 Suppl 11: S33-6, 1994.;Tan, E. M., Cell. 67: 841-2, 1991; Abrams, M. B., Bednarek, K. T., Bogoch, S., Bogoch, E. S., Dardik, H. J., Dowden, R., Fox, S. C., Goins, E. E., Goodfried, G., Herrman, R. A., and et al., Cancer Detect Prev. 18: 65-78, 1994. Amagai, M., Klaus-Kovtun, V., and Stanley, J. R., Cell. 67: 869-77, 1991).

### SUMMARY OF THE INVENTION

Applicants have identified cellular components that may trigger immune responses in cancer patients. In particular, Applicants screened a breast carcinoma cDNA expression library with serum from breast cancer patients and identified cellular proteins that trigger autoantibody production. The proteins include protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); and, plakophilin 4 (PKP4) (Genbank NM-003628).

A novel nucleic acid molecule was also identified by Applicants in the immunoscreen of the breast carcinoma cDNA library. The novel nucleic acid molecules of the invention are herein referred to as “breast cancer tumor associated gene”, “bta ”, or “bta gene ”. A polypeptide encoded by a nucleic acid molecule of the invention is referred to herein as “BTA Protein(s)”.

The cellular proteins that trigger autoantibody production identified in the immunoscreen including the proteins referenced above and BTA Related Proteins described herein are referred to herein as “tumor associated protein(s)”.

Broadly stated the present invention relates to an isolated nucleic acid molecule of at least 30 nucleotides which hybridizes to one or more of SEQ. ID. NO. 1 to 8, or the complement of one or more of SEQ ID NOs. 1 to 8, under stringent hybridization conditions.

The invention also contemplates a nucleic acid molecule comprising a sequence encoding a truncation of a BTA Protein, an analog, or a homolog of a BTA Protein or a truncation thereof (BTA Protein and truncations, analogs and homologs of BTA Protein are also collectively referred to herein as “BTA Related Proteins”).

The nucleic acid molecules of the invention may be inserted into an appropriate expression vector, i.e. a vector that contains the necessary elements for the transcription and translation of the inserted coding sequence. Accordingly, recombinant expression vectors adapted for transformation of a host cell may be constructed which comprise a nucleic acid molecule of the invention and one or more transcription and translation elements linked to the nucleic acid molecule.

In accordance with an aspect of the invention, a vector is provided comprising a DNA molecule with a nucleotide sequence encoding at least one epitope of a BTA Protein, and suitable regulatory sequences to allow expression in a host cell.

The recombinant expression vector can be used to prepare transformed host cells expressing BTA Related Proteins. Therefore, the invention further provides host cells containing a vector of the invention. The invention also contemplates transgenic non-human mammals whose germ cells and somatic cells contain a vector comprising a nucleic acid molecule of the invention, in particular one which encodes an analog of a BTA Protein, or a truncation of a BTA Protein.

The invention further provides a method for preparing BTA Related Proteins utilizing the purified and isolated nucleic acid molecules of the invention. In an embodiment a method for preparing a BTA Related Protein is provided comprising (a) transferring a recombinant expression vector of the invention into a host cell; (b) selecting transformed host cells from untransformed host cells; (c) culturing a selected transformed host cell under conditions which allow expression of the BTA Related Protein; and (d) isolating the BTA Related Protein.

The invention further broadly contemplates an isolated BTA Protein. An aspect of the invention provides an amino acid sequence deduced from the protein coding region of a nucleic acid molecule of the invention.

The tumor associated proteins, in particular BTA Related Proteins of the invention may be conjugated with other molecules, such as proteins, to prepare fusion proteins. This may be accomplished, for example, by the synthesis of N-terminal or C-terminal fusion proteins.

The invention further contemplates antibodies having specificity against an epitope of a tumor associated protein described herein, in particular a BTA Related Protein of the invention. Antibodies may be labeled with a detectable substance and used to detect the proteins of the invention in tissues and cells. Antibodies may have particular use in therapeutic applications, for example to react with tumor cells, and in conjugates and immunotoxins as target selective carriers of various agents which have antitumor effects including chemotherapeutic drugs, toxins, immunological response modifiers, enzymes, and radioisotopes.

The invention also permits the construction of nucleotide probes that are unique to nucleic acid molecules encoding tumor associated proteins described herein, in particular a BTA Protein. In an aspect of the invention a probe is provided comprising a nucleic acid molecule of the invention, or a nucleic acid sequence encoding a tumor associated protein described herein, or a part thereof. A probe may be labeled, for example, with a detectable substance and it may be used to select from a mixture of nucleotide sequences a nucleic acid molecule encoding a tumor associated protein, in particular a BTA Related Protein. A probe may be used to mark tumors, in particular breast tumors.

The invention also provides antisense nucleic acid molecules e.g. by production of a mRNA or DNA strand in the reverse orientation to a sense molecule. An antisense nucleic acid molecule may be used to suppress the growth of a tumor associated protein (e,g. BTA Protein) expressing (e.g. cancerous) cell.

The invention still further provides a method for identifying a substance which binds to a tumor associated protein (e.g. BTA Related Protein) comprising reacting the protein with at least one substance which potentially can bind with the protein, under conditions which permit the formation of complexes between the substance and protein and detecting binding. Binding may be detected by assaying for complexes, for free substance, or for non-complexed protein. The invention also contemplates methods for identifying substances that bind to other intracellular proteins that interact with a tumor associated protein (e.g. BTA Related Protein). Methods can also be utilized which identify compounds which bind to tumor associated protein gene regulatory sequences (e.g. promoter sequences).

Still further the invention provides a method for evaluating a compound for its ability to modulate the biological activity of a tumor associated protein (e.g. BTA Related Protein). For example a substance which inhibits or enhances the interaction of the protein and a substance which binds to the protein may be evaluated. In an embodiment, the method comprises providing a known concentration of a tumor associated protein (e.g. BTA Related Protein), with a substance which binds to the protein and a test compound under conditions which permit the formation of complexes between the substance and protein, and removing and/or detecting complexes.

In other embodiments, the invention provides a method for identifying inhibitors of a BTA Related Protein interaction, comprising

(a) providing a reaction mixture including the BTA Related Protein and a substance that binds to the BTA Related Protein, or at least a portion of each which interact;

(b) contacting the reaction mixture with one or more test compounds;

(c) identifying compounds which inhibit the interaction of the BTA Related Protein and substance.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as those isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a BTA Related Protein and a substance that binds to the protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Compounds which modulate the biological activity of a tumor associated protein (e.g. BTA Related Protein) may also be identified using the methods of the invention by comparing the pattern and level of expression of the protein in tissues and cells, in the presence, and in the absence of the compounds.

Antibodies, nucleic acid molecules, and antisense nucleic acid molecules of the invention and substances and compounds identified using the methods of the invention may be used to modulate the biological activity of a tumor associated protein described herein, and they may be used in the treatment of conditions such as cancer (particularly breast cancer) in a patient. Accordingly, the antibodies, nucleic acid molecules, antisense molecules, substances and compounds may be formulated into compositions for administration to individuals suffering from disorders such as cancer (particularly breast cancer) in a patient. In particular, the antibodies, antisense nucleic acid molecules, substances and compounds may be used to treat patients who have a tumor associated protein in, or on, their cancer cells.

Therefore, the present invention also relates to a composition comprising one or more of a tumor associated protein described herein, a peptide thereof, a nucleic acid molecule, or a substance or compound identified using the methods of the invention, and a pharmaceutically acceptable carrier, excipient or diluent. A method for treating or preventing a disorder such as cancer (particularly breast cancer) in a patient is also provided comprising administering to a patient in need thereof, a tumor associated protein (e.g. BTA Related Protein), a nucleic acid molecule, or a composition of the invention.

Another aspect of the invention is the use of a tumor associated protein (e.g. BTA Protein), peptides derived therefrom, or chemically produced (synthetic) peptides, or any combination of these molecules, for use in the preparation of vaccines to prevent cancer and/or to treat cancer, in particular to prevent and/or treat cancer in patients who have a tumor associated protein detected on their cells. These vaccine preparations may also be used to prevent patients from having tumors, in particular breast tumors, prior to their occurrence.

The invention broadly contemplates vaccines for stimulating or enhancing in a subject to whom the vaccine is administered production of antibodies directed against a tumor associated protein (e.g. BTA Protein).

The invention also provides a method for stimulating or enhancing in a subject production of antibodies directed against a tumor associated protein (e.g. BTA Protein). The method comprises administering to the subject a vaccine of the invention in a dose effective for stimulating or enhancing production of the antibodies.

The invention further provides methods for treating, preventing, or delaying recurrence of cancer. The methods comprise administering to the subject a vaccine of the invention in a dose effective for treating, preventing, or delaying recurrence of cancer.

In an embodiment of the invention, a method is provided for diagnosing and monitoring cancer mediated by a tumor associated protein by determining the presence of a nucleic acid molecule encoding the tumor associated protein or the presence of the tumor associated protein, wherein the tumor associated protein is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by a bta nucleic acid molecule.

In another embodiment of the invention, a method is provided for preventing or treating a condition mediated by a tumor associated protein comprising administering an effective amount of an antibody specific for the tumor associated protein, wherein the tumor associated protein is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a BTA Related Protein.

In a further embodiment, a vaccine is provided to prevent cancer and/or to treat cancer comprising a tumor associated protein which is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL 1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by a bta nucleic acid molecule, peptides derived from the proteins, or synthetic peptides thereof, or any combination of these molecules.

In a still further embodiment, a method is provided to prevent cancer or to treat cancer in subjects who have a tumor associated protein on their cells comprising administering a vaccine for stimulating or enhancing in the subjects antibodies directed against the tumor associated protein wherein the tumor associated protein is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by a bta nucleic acid molecule.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the constructed 608-1 cDNA sequence (with sequence identified by immunoscreening, ESTs AW975482, AW410740, and N73248). The novel sequence identified by the immunoscreening method is in bold. The EST sequences are in regular font.

FIG. 2 shows the genomic organization of the constructed 608-1 cDNA sequence. This sequence was divided into four exons (capital letters) with three intervening introns (lower case letters). Dotted lines represent sequences that are not shown. For detailed sequence information see Genbank submission AC009542. The splice donor and acceptor sites are shown in bold letters. The intron/exon lengths are as indicated. The arrows represent the unknown cDNA sequence.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y.); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

1. BTA Nucleic Acid Molecules of the Invention

As hereinbefore mentioned, the invention provides an isolated nucleic acid molecule having a sequence encoding a BTA Protein. The term “isolated” refers to a nucleic acid substantially free of cellular material or culture medium when produced by recombinant DNA techniques, or chemical reactants, or other chemicals when chemically synthesized. An “isolated” nucleic acid may also be free of sequences which naturally flank the nucleic acid (i.e., sequences located at the 5′ and 3′ ends of the nucleic acid molecule) from which the nucleic acid is derived. The term “nucleic acid” is intended to include DNA and RNA and can be either double stranded or single stranded. A nucleic acid molecule that is introduced into an organism by transformation, genetic manipulation, or any other recombinant method is “isolated” even if it is still present in an organism, which may be living or non-living. Preferably, an isolated nucleic acid molecule is at least 60% free, more preferably at least 75% free, and most preferably at least 90%, 95%, or 98% free from other components with which they are naturally associated.

In an aspect of the invention, a nucleic acid molecule comprises a nucleic acid sequence of one or more of SEQ.ID.NOs. 1 to 8.

In an embodiment, the invention provides an isolated nucleic acid molecule which comprises:

(i) a nucleic acid sequence comprising the sequence of one or more of SEQ.ID.NOs. 1 to 8 wherein T can also be U;

(ii) nucleic acid sequences complementary to (i), preferably complementary to the full nucleic acid sequence of one or more of SEQ.ID.NOs. 1 to 8;

(iii) a nucleic acid capable of hybridizing under stringent conditions to a nucleic acid of (i) or (ii) and preferably having at least 18, 20, 25, 30, 35, or 40 nucleotides; or

(iv) a nucleic acid molecule differing from any of the nucleic acids of (i) to (iii) in codon sequences due to the degeneracy of the genetic code.

The invention includes nucleic acid sequences complementary to a full nucleic acid sequence of any one of SEQ.ID. NOs. 1 to 8. The term “complementary” refers to the natural binding of polynucleotides under permissive salt and temperature conditions by base-pairing. For example, the sequence “A—G—T” binds to the complementary sequence “T—C—A”. Complementarity between two single-stranded molecules may be “partial”, in which only some of the nucleic acids bind, or it may be complete when total complementarity exists between the single stranded molecules.

The invention includes nucleic acid molecules having substantial sequence identity or homology to nucleic acid sequences of the invention. Preferably, the nucleic acids have substantial sequence identity for example at least 20%, 30%, 40%, 45%, 50%, 55%, 60%, 65%, 70%, 75%, 80%, or 85% nuclic acid identity; more preferably 90% nucleic acid identity; and most preferably at least 95%, 96%, 97%, 98%, or 99% sequence identity. “Identity” as known in the art and used herein, is a relationship between two or more amino acid sequences or two or more nucleic acid sequences, as determined by comparing the sequences. It also refers to the degree of sequence relatedness between amino acid or nucleic acid sequences, as the case may be, as determined by the match between strings of such sequences. Identity and similarity are well known terms to skilled artisans and they can be calculated by conventional methods (for example see Computational Molecular Biology, Lesk, A. M. ed., Oxford University Press, New York, 1988; Biocomputing: Informatics and Genome Projects, Smith, D. W. ed., Academic Press, New York, 1993; Computer Analysis of Sequence Data, Part I, Griffin, A. M. and Griffin, H. G. eds., Humana Press, New Jersey, 1994; Sequence Analysis in Molecular Biology, von Heinje, G. Acadmeic Press, 1987; and Sequence Analysis Primer, Gribskov, M. and Devereux, J. eds. M. Stockton Press, New York, 1991, Carillo, H. and Lipman, D., SIAM J. Applied Math. 48:1073, 1988). Methods which are designed to give the largest match between the sequences are generally preferred. Methods to determine identity and similarity are codified in publicly available computer programs including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990).

Parameters for comparison of nucleic acid sequences include the following: (1) Algorithm: Needleman and Wunsch, J. Mol Biol. 48: 443-453 (1970); (2) Comparison matrix: matches=+10, mismatch=0; (3) Gap Penalty: 50; and (4) Gap Length Penalty: 3. The “gap” program from Genetics Computer Group, Madison, Wis. is a publicly available program with these default parameters for nucleic acid comparisons.

Isolated nucleic acid molecules encoding a BTA Protein, and having a sequence which differs from a nucleic acid sequence of the invention due to degeneracy in the genetic code are also within the scope of the invention. Such nucleic acids encode functionally equivalent proteins (e.g. a BTA Protein) but differ in sequence from the sequence of a BTA Protein due to degeneracy in the genetic code. As one example, DNA sequence polymorphisms within the nucleotide sequence of a bta gene may result in silent mutations which do not affect the amino acid sequence. Variations in one or more nucleotides may exist among individuals within a population due to natural allelic variation. Any and all such nucleic acid variations are within the scope of the invention. DNA sequence polymorphisms may also occur which lead to changes in the amino acid sequence of a BTA Protein. These amino acid polymorphisms are also within the scope of the present invention.

Another aspect of the invention provides a nucleic acid molecule which hybridizes under stringent conditions, preferably high stringency conditions to a nucleic acid molecule of one or more of SEQ.ID.NO. 1 to 8. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0×sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

It will be appreciated that the invention includes nucleic acid molecules encoding a BTA Related Protein including truncations of a BTA Protein, and analogs of a BTA Protein as described herein. The truncated nucleic acids or nucleic acid fragments may correspond to a sequence of one of SEQ ID Nos. 2 to 8 inclusive. It will further be appreciated that variant forms of the nucleic acid molecules of the invention which arise by alternative splicing of an mRNA corresponding to a cDNA of the invention are encompassed by the invention.

An isolated nucleic acid molecule of the invention which comprises DNA can be isolated by preparing a labelled nucleic acid probe based on all or part of a nucleic acid sequence of the invention. The labeled nucleic acid probe is used to screen an appropriate DNA library (e.g. a cDNA or genomic DNA library). For example, a cDNA library can be used to isolate a cDNA of the invention by screening the library with the labeled probe using standard techniques. Alternatively, a genomic DNA library can be similarly screened to isolate a genomic clone encompassing a gene encoding a BTA Related Protein. Nucleic acids isolated by screening of a cDNA or genomic DNA library can be sequenced by standard techniques.

An isolated nucleic acid molecule of the invention which is DNA can also be isolated by selectively amplifying a nucleic acid molecule of the invention using the polymerase chain reaction (PCR) methods and cDNA or genomic DNA. It is possible to design synthetic oligonucleotide primers from the nucleotide sequence of the invention for use in PCR. A nucleic acid can be amplified from cDNA or genomic DNA using these oligonucleotide primers and standard PCR amplification techniques. The nucleic acid so amplified can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

An isolated nucleic acid molecule of the invention which is RNA can be isolated by cloning a cDNA into an appropriate vector which allows for transcription of the cDNA to produce an RNA molecule of the invention. For example, a cDNA can be cloned downstream of a bacteriophage promoter, (e.g. a T7 promoter) in a vector, cDNA can be transcribed in vitro with T7 polymerase, and the resultant RNA can be isolated by conventional techniques.

Nucleic acid molecules of the invention may be chemically synthesized using standard techniques.

Methods of chemically synthesizing polydeoxynucleotides are known, including but not limited to solid-phase synthesis which, like peptide synthesis, has been fully automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071).

Determination of whether a particular nucleic acid molecule encodes a BTA Related Protein can be accomplished by expressing the cDNA in an appropriate host cell by standard techniques, and testing the expressed protein in the methods described herein. A cDNA can be sequenced by standard techniques, such as dideoxynucleotide chain termination or Maxam-Gilbert chemical sequencing, to determine the nucleic acid sequence and the predicted amino acid sequence of the encoded protein.

The initiation codon and untranslated sequences of a nucleic acid molecule of the invention may be determined using computer software designed for the purpose, such as PC/Gene (IntelliGenetics Inc., Calif.). The intron-exon structure and the transcription regulatory sequences of a gene of the invention may be confirmed by using a nucleic acid molecule of the invention to probe a genomic DNA clone library. Regulatory elements can be identified using standard techniques. The function of the elements can be confirmed by using these elements to express a reporter gene such as the lacZ gene that is operatively linked to the elements. These constructs may be introduced into cultured cells using conventional procedures or into non-human transgenic animal models. In addition to identifying regulatory elements in DNA, such constructs may also be used to identify nuclear proteins interacting with the elements, using techniques known in the art.

In a particular embodiment of the invention, the nucleic acid molecules isolated using the methods described herein are mutant BTA gene alleles. The mutant alleles may be isolated from individuals either known or proposed to have a genotype which contributes to the symptoms of a disorder involving a BTA Related Protein. Mutant alleles and mutant allele products may be used in therapeutic and diagnostic methods described herein. For example, a cDNA of a mutant bta gene may be isolated using PCR as described herein, and the DNA sequence of the mutant allele may be compared to the normal allele to ascertain the mutation(s) responsible for the loss or alteration of function of the mutant gene product. A genomic library can also be constructed using DNA from an individual suspected of or known to carry a mutant allele, or a cDNA library can be constructed using RNA from tissue known, or suspected to express the mutant allele. A nucleic acid encoding a normal BTA gene or any suitable fragment thereof, may then be labeled and used as a probe to identify the corresponding mutant allele in such libraries. Clones containing mutant sequences can be purified and subjected to sequence analysis. In addition, an expression library can be constructed using cDNA from RNA isolated from a tissue of an individual known or suspected to express a mutant bta allele. Gene products made by the putatively mutant tissue may be expressed and screened, for example using antibodies specific for a BTA Related Protein as described herein. Library clones identified using the antibodies can be purified and subjected to sequence analysis.

The sequence of a nucleic acid molecule of the invention, or a fragment of the molecule, may be inverted relative to its normal presentation for transcription to produce an antisense nucleic acid molecule. An antisense nucleic acid molecule may be constructed using chemical synthesis and enzymatic ligation reactions using procedures known in the art.

2. BTA Related Proteins of the Invention

The invention contemplates a BTA Protein deduced from the protein coding region of a nucleic acid molecule of the invention. The proteins of the present invention also include truncations of a BTA Protein, analogs of a BTA Protein, and proteins having sequence identity or similarity to a BTA Protein, and truncations thereof as described herein (i.e. BTA Related Proteins).

Truncated proteins may comprise peptides of between 3 and 70 amino acid residues, ranging in size from a tripeptide to a 70 mer polypeptide. The truncated proteins may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated proteins may have a carboxyl group, an amido group, a T-butyloxycarbonyl group, or a macromolecule including but not limited to lipid-fatty acid conjugates, polyethylene glycol, or carbohydrates at the carboxy terminal end.

The proteins of the invention may also include analogs of a BTA Protein, and/or truncations thereof as described herein, which may include, but are not limited to a BTA protein, containing one or more amino acid substitutions, insertions, and/or deletions. Amino acid substitutions may be of a conserved or non-conserved nature. Conserved amino acid substitutions involve replacing one or more amino acids of a BTA Protein amino acid sequence with amino acids of similar charge, size, and/or hydrophobicity characteristics. When only conserved substitutions are made the resulting analog is preferably functionally equivalent to a BTA Protein. Non-conserved substitutions involve replacing one or more amino acids of the BTA Protein amino acid sequence with one or more amino acids that possess dissimilar charge, size, and/or hydrophobicity characteristics.

One or more amino acid insertions may be introduced into a BTA Protein. Amino acid insertions may consist of single amino acid residues or sequential amino acids ranging from 2 to 15 amino acids in length.

Deletions may consist of the removal of one or more amino acids, or discrete portions from a BTA Protein sequence. The deleted amino acids may or may not be contiguous. The lower limit length of the resulting analog with a deletion mutation is about 10 amino acids, preferably 20 to 40 amino acids.

The proteins of the invention include proteins with sequence identity or similarity to a BTA Protein and/or truncations thereof as described herein. Such BTA Proteins include proteins whose amino acid sequences are comprised of the amino acid sequences of BTA Protein regions from other species that hybridize under selected hybridization conditions (see discussion of stringent hybridization conditions herein) with a probe used to obtain a BTA Protein. These proteins will generally have the same regions which are characteristic of a BTA Protein. Preferably a protein will have substantial sequence identity for example, about 20%, 30%, 40%, 50%, 60%, 65%, 70%, 75%, 80%, or 85% identity, preferably 90% identity, more preferably at least 95%, 96%, 97%, 98%, or 99% identity, and most preferably 98%. A percent amino acid sequence homology, similarity or identity is calculated as the percentage of aligned amino acids that match the reference sequence using known methods as described herein.

The invention also contemplates isoforms of the proteins of the invention. An isoform contains the same number and kinds of amino acids as a protein of the invention, but the isoform has a different molecular structure. Isoforms contemplated by the present invention preferably have the same properties as a protein of the invention as described herein.

The present invention also includes BTA Related Proteins conjugated with a selected protein (e.g. therapeutic agents), or a marker protein (see below) to produce fusion proteins. Immunogenic portions of a BTA Protein and a BTA Protein Related Protein are within the scope of the invention.

A BTA Related Protein of the invention may be prepared using recombinant DNA methods. Accordingly, a nucleic acid molecule of the present invention may be incorporated in a known manner into an appropriate expression vector which ensures good expression of the protein. Possible expression vectors include but are not limited to cosmids, plasmids, or modified viruses (e.g. replication defective retroviruses, adenoviruses and adeno-associated viruses), so long as the vector is compatible with the host cell used.

The invention therefore contemplates a recombinant expression vector of the invention containing a nucleic acid molecule of the invention, and the necessary regulatory sequences for the transcription and translation of the inserted protein-sequence. Suitable regulatory sequences may be derived from a variety of sources, including bacterial, fungal, viral, mammalian, or insect genes [For example, see the regulatory sequences described in Goeddel, Gene Expression Technology: Methods in Enzymology 185, Academic Press, San Diego, Calif. (1990)]. Selection of appropriate regulatory sequences is dependent on the host cell chosen as discussed below, and may be readily accomplished by one of ordinary skill in the art. The necessary regulatory sequences may be supplied by the native gene and/or its flanking regions.

The invention further provides a recombinant expression vector comprising a DNA nucleic acid molecule of the invention cloned into the expression vector in an antisense orientation. That is, the DNA molecule is linked to a regulatory sequence in a manner which allows for expression, by transcription of the DNA molecule, of an RNA molecule which is antisense to the nucleic acid sequence of a protein of the invention or a fragment thereof. Regulatory sequences linked to the antisense nucleic acid can be chosen which direct the continuous expression of the antisense RNA molecule in a variety of cell types, for instance a viral promoter and/or enhancer, or regulatory sequences can be chosen which direct tissue or cell type specific expression of antisense RNA.

The recombinant expression vectors of the invention may also contain a marker gene which facilitates the selection of host cells transformed or transfected with a recombinant molecule of the invention. Examples of marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The markers can be introduced on a separate vector from the nucleic acid of interest.

The recombinant expression vectors may also contain genes that encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

The recombinant expression vectors may be introduced into host cells to produce a transformant host cell. “Transformant host cells” include host cells which have been transformed or transfected with a recombinant expression vector of the invention. The terms “transformed with”, “transfected with”, “transformation” and “transfection” encompass the introduction of a nucleic acid (e.g. a vector) into a cell by one of many standard techniques. Prokaryotic cells can be transformed with a nucleic acid by, for example, electroporation or calcium-chloride mediated transformation. A nucleic acid can be introduced into mammalian cells via conventional techniques such as calcium phosphate or calcium chloride co-precipitation, DEAE-dextran-mediated transfection, lipofectin, electroporation or microinjection. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the proteins of the invention may be expressed in bacterial cells such as E. coli, insect cells (using baculovirus), yeast cells, or mammalian cells. Other suitable host cells can be found in Goeddel, Gene Expression Technology: Methods in Enzymology 185, Academic Press, San Diego, Calif. (1991).

A host cell may also be chosen which modulates the expression of an inserted nucleic acid sequence, or modifies (e.g. glycosylation or phosphorylation) and processes (e.g. cleaves) the protein in a desired fashion. Host systems or cell lines may be selected which have specific and characteristic mechanisms for post-translational processing and modification of proteins. For example, eukaryotic host cells including CHO, VERO, BHK, HeLA, COS, MDCK, 293, 3T3, and WI38 may be used. For long-term high-yield stable expression of the protein, cell lines and host systems which stably express the gene product may be engineered.

Host cells and in particular cell lines produced using the methods described herein may be particularly useful in screening and evaluating compounds that modulate the activity of a protein or nucleic acid of the invention.

The proteins of the invention may also be expressed in non-human transgenic animals including but not limited to mice, rats, rabbits, guinea pigs, micro-pigs, goats, sheep, pigs, non-human primates (e.g. baboons, monkeys, and chimpanzees) [see Hammer et al. (Nature 315:680-683, 1985), Palmiter et al. (Science 222:809-814, 1983), Brinster et al. (Proc Natl. Acad. Sci USA 82:44384442, 1985), Palmiter and Brinster (Cell. 41:343-345, 1985) and U.S. Pat. No. 4,736,866)]. Procedures known in the art may be used to introduce a nucleic acid molecule of the invention into animals to produce the founder lines of transgenic animals. Such procedures include pronuclear microinjection, retrovirus mediated gene transfer into germ lines, gene targeting in embryonic stem cells, electroporation of embryos, and sperm-mediated gene transfer.

The present invention contemplates a transgenic animal that carries the bta gene in all their cells, and animals which carry the transgene in some but not all their cells. The transgene may be integrated as a single transgene or in concatamers. The transgene may be selectively introduced into and activated in specific cell types (See for example, Lasko et al, 1992 Proc. Natl. Acad. Sci. USA 89: 6236). The transgene may be integrated into the chromosomal site of the endogenous gene by gene targeting. The transgene may be selectively introduced into a particular cell type inactivating the endogenous gene in that cell type (See Gu et al Science 265: 103-106).

The expression of a recombinant BTA Related Protein in a transgenic animal may be assayed using standard techniques. Initial screening may be conducted by Southern Blot analysis, or PCR methods to analyze whether the transgene has been integrated. The level of mRNA expression in the tissues of transgenic animals may also be assessed using techniques including Northern blot analysis of tissue samples, in situ hybridization, and RT-PCR. Tissue may also be evaluated immunocytochemically using antibodies against a BTA Protein.

BTA Related Proteins of the invention may also be prepared by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart).

N-terminal or C-terminal fusion proteins comprising a BTA Related Protein of the invention conjugated with other molecules, such as proteins, may be prepared by fusing, through recombinant techniques, the N-terminal or C-terminal of a BTA Related Protein, and the sequence of a selected protein or marker protein with a desired biological function. The resultant fusion proteins contain a BTA Protein fused to the selected protein or marker protein as described herein. Examples of proteins which may be used to prepare fusion proteins include immunoglobulins, glutathione-S-transferase (GST), hemagglutinin (HA), and truncated myc.

3. Antibodies

The tumor associated proteins, in particular BTA Related Proteins, can be used to prepare antibodies specific for the proteins. Antibodies can be prepared which bind a distinct epitope in an unconserved region of the protein. An unconserved region of the protein is one that does not have substantial sequence homology to other proteins. A region from a conserved region such as a well-characterized domain can also be used to prepare an antibody to a conserved region of a tumor associated protein. Antibodies having specificity for a tumor associated protein may also be raised from fusion proteins created by expressing fusion proteins in bacteria as described herein.

The invention can employ for example, intact monoclonal or polyclonal antibodies, and immunologically active fragments (e.g. a Fab, (Fab)₂ fragment, or Fab expression library fragments and epitope-binding fragments thereof), an antibody heavy chain, and antibody light chain, a genetically engineered single chain Fv molecule (Ladner et al, U.S. Pat. No. 4,946,778), humanized antibodies, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

4. Applications

Nucleic acid molecules encoding tumor associated proteins (e.g. BTA Related Proteins), tumor associated proteins (e.g. BTA Related Proteins), and antibodies against the tumor associated proteins may be used in the prognostic and diagnostic evaluation of disorders involving the tumor associated proteins (e.g. BTA Related Protein), and the identification of subjects with a predisposition to such disorders (Section 4.1.1 and 4.1.2).

Methods for detecting nucleic acid molecules encoding tumor associated proteins and the tumor associated proteins, can be used to monitor disorders involving a tumor associated protein (e.g. cancer, in particular breast cancer) by detecting the proteins and nucleic acid molecules.

In an embodiment of the invention, a method is provided for detecting the expression of a cancer marker in a patient comprising:

(a) taking a sample derived from a patient; and

(b) detecting in the sample a nucleic acid sequence encoding a tumor associated protein (e.g.BTA Protein) or a protein product encoded by a tumor associated protein nucleic acid sequence (e.g. bta).

In a particular embodiment of the invention, the nucleic acid molecules, tumor associated proteins, and antibodies of the invention may be used in the diagnosis and staging of cancer, in particular breast cancer.

The applications of the present invention also include methods for the identification of compounds that modulate the biological activity of a tumor associated protein (e.g. BTA Related Protein) (Section 4.2). The compounds, antibodies etc. may be used for the treatment of disorders involving a tumor associated protein (e.g. BTA Related Protein) (Section 4.3). It would also be apparent to one skilled in the art that the methods described herein may be used to study the developmental expression of tumor associated proteins (e.g. BTA Related Proteins) and, accordingly, will provide further insight into the role of the proteins.

4.1 Diagnostic Methods

A variety of methods can be employed for the diagnostic and prognostic evaluation of disorders involving a tumor associated protein (e.g. BTA Related Protein), and the identification of subjects with a predisposition to such disorders. Such disorders include cancer, particularly breast cancer. Such methods may, for example, utilize nucleic acid molecules encoding tumor associated proteins, and fragments thereof, and antibodies directed against tumor associated proteins, including peptide fragments. In particular, the nucleic acids and antibodies may be used, for example, for: (1) the detection of the presence of mutations in genes encoding a tumor associated protein (e.g. bta), or the detection of either over- or under-expression of a tumor associated protein mRNA relative to a non-disorder state, or the qualitative or quantitative detection of alternatively spliced forms of tumor associated protein transcripts which may correlate with certain conditions or susceptibility toward such conditions; and (2) the detection of either an over- or an under-abundance of tumor associated proteins relative to a non- disorder state or the presence of a modified (e.g., less than full length) tumor associated protein which correlates with a disorder state, or a progression toward a disorder state.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein may be performed by utilizing pre-packaged diagnostic kits comprising at least one specific nucleic acid or antibody described herein, which may be conveniently used, e.g., in clinical settings, to screen and diagnose patients and to screen and identify those individuals exhibiting a predisposition to developing a disorder.

Nucleic acid-based detection techniques are described, below, in Section 4.1.1. Peptide detection techniques are described, below, in Section 4.1.2. The samples that may be analyzed using the methods of the invention include those which are known or suspected to express nucleic acids encoding tumor associated proteins (e.g. bta) or contain tumor associated proteins (e.g. BTA Related Proteins). The samples may be derived from a patient or a cell culture, and include but are not limited to biological fluids, tissue extracts, freshly harvested cells, and lysates of cells which have been incubated in cell cultures.

Oligonucleotides or longer fragments derived from any of the nucleic acid molecules encoding tumor associated proteins may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I995) PCT application WO95/35505; Heller, R. A. et al. (1997) Proc. Natl. Acad. Sci. 94:2150-2155; and Heller, M. J. et al. (1997) U.S. Pat. No. 5,605,662.)

4.1.1 Methods for Detecting Nucleic Acid Molecules

Those skilled in the art can construct nucleotide probes for use in the detection of nucleic acid sequences encoding tumor associated proteins in samples. Suitable probes include nucleic acid molecules based on nucleic acid sequences encoding at least 5 sequential amino acids from regions of a tumor associated protein (e.g. BTA Related Protein), preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect genes, preferably in human cells, that encode tumor associated proteins (e.g. BTA Related Proteins). The nucleotide probes may also be useful in the diagnosis of disorders involving a tumor associated protein (e.g. BTA Related Protein); in monitoring the progression of such disorders; or monitoring a therapeutic treatment. In an embodiment, the probes are used in the diagnosis of, and in monitoring the progression of cancer, preferably breast cancer.

The probe may be used in hybridization techniques to detect genes that encode tumor associated proteins (e.g. BTA Related Proteins). The technique generally involves contacting and incubating nucleic acids (e.g. recombinant DNA molecules, cloned genes) obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules may involve the amplification of specific gene sequences using an amplification method such as PCR, followed by the analysis of the amplified molecules using techniques known to those skilled in the art. Suitable primers can be routinely designed by one of skill in the art.

Genomic DNA may be used in hybridization or amplification assays of biological samples to detect abnormalities involving the structure of genes encoding tumor associated proteins (e.g. bta), including point mutations, insertions, deletions, and chromosomal rearrangements. For example, direct sequencing, single stranded conformational polymorphism analyses, heteroduplex analysis, denaturing gradient gel electrophoresis, chemical mismatch cleavage, and oligonucleotide hybridization may be utilized.

Genotyping techniques known to one skilled in the art can be used to type polymorphisms that are in close proximity to the mutations of a tumor associated protein gene. The polymorphisms may be used to identify individuals in families that are likely to carry mutations. If a polymorphism exhibits linkage disequalibrium with mutations in a tumor associated protein gene, it can also be used to screen for individuals in the general population likely to carry mutations. Polymorphisms which may be used include restriction fragment length polymorphisms (RFLPs), single-base polymorphisms, and simple sequence repeat polymorphisms (SSLPs).

A probe of the invention may be used to directly identify RFLPs. A probe or primer of the invention can additionally be used to isolate genomic clones such as YACs, BACs, PACs, cosmids, phage or plasmids. The DNA in the clones can be screened for SSLPs using hybridization or sequencing procedures.

Hybridization and amplification techniques described herein may be used to assay qualitative and quantitative aspects of expression of genes encoding tumor associated proteins. For example, RNA may be isolated from a cell type or tissue known to express a gene encoding a tumor associated protein, and tested utilizing the hybridization (e.g. standard Northern analyses) or PCR techniques referred to herein. The techniques may be used to detect differences in transcript size which may be due to normal or abnormal alternative splicing. The techniques may be used to detect quantitative differences between levels of full length and/or alternatively splice transcripts detected in normal individuals relative to those individuals exhibiting symptoms of a disorder involving a tumor associated protein (e.g. BTA Related Protein) or gene.

The primers and probes may be used in the above described methods in situ i.e directly on tissue sections (fixed and/or frozen) of patient tissue obtained from biopsies or resections.

4.1.2 Methods for Detecting Tumor Associated Proteins

Antibodies specifically reactive with a tumor associated protein (e.g. BTA Related Protein), or derivatives, such as enzyme conjugates or labeled derivatives, may be used to detect tumor associated proteins in various samples (e.g. biological materials). They may be used as diagnostic or prognostic reagents and they may be used to detect abnormalities in the level of protein expression, or abnormalities in the structure, and/or temporal, tissue, cellular, or subcellular location of a tumor associated protein (e.g. BTA Related Protein). Antibodies may also be used to screen potentially therapeutic compounds in vitro to determine their effects on disorders involving a tumor associated protein (e.g. BTA Related Protein), and other conditions. In vitro immunoassays may also be used to assess or monitor the efficacy of particular therapies. The antibodies of the invention may also be used in vitro to determine the level of expression of a gene encoding a tumor associated protein (e.g. bta) in cells genetically engineered to produce a tumor associated protein.

The antibodies may be used in any known immunoassays which rely on the binding interaction between an antigenic determinant of a tumor associated protein (e.g. BTA Related Protein) and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify a tumor associated protein (e.g. BTA Related Protein) in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect a tumor associated protein (e.g. BTA Related Protein), to localize it to particular cells and tissues, and to specific subcellular locations, and to quantitate the level of expression.

Cytochemical techniques known in the art for localizing antigens using light and electron microscopy may be used to detect a tumor associated protein (e.g BTA Related Protein). Generally, an antibody of the invention may be labeled with a detectable substance and a tumor associated protein may be localised in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against a tumor associated protein. By way of example, if the antibody having specificity against a tumor associated protein is a rabbit IgG antibody, the second antibody may be goat anti-rabbit gammaglobulin labeled with a detectable substance as described herein.

Where a radioactive label is used as a detectable substance, a tumor associated protein (e.g BTA Related Protein) may be localized by radioautography. The results of radioautography may be quantitated by determining the density of particles in the radioautographs by various optical methods, or by counting the grains.

In an embodiment, the invention contemplates a method for monitoring the progression of cancer (e.g. breast cancer) in an individual, comprising:

(a) contacting an amount of an antibody which binds to a BTA Related Protein, with a sample from the individual so as to form a binary complex comprising the antibody and BTA Related Protein in the sample;

(b) determining or detecting the presence or amount of complex formation in the sample;

(c) repeating steps (a) and (b) at a point later in time; and

(d) comparing the result of step (b) with the result of step (c), wherein a difference in the amount of complex formation is indicative of the progression of the cancer in said individual.

Labeled antibodies against tumor associated proteins may be used in locating tumor tissue in patients undergoing surgery i.e. in imaging. Typically for in vivo applications, antibodies are labeled with radioactive labels (e.g. iodine-123, iodine-125, iodine-131, gallium-67, technetium-99, and indium-111). Labeled antibody preparations may be administered to a patient intravenously in an appropriate carrier at a time several hours to four days before the tissue is imaged. During this period unbound fractions are cleared from the patient and the only remaining antibodies are those associated with tumor tissue. The presence of the isotope is detected using a suitable gamma camera. The labeled tissue can be correlated with known markers on the patient's body to pinpoint the location of the tumor for the surgeon.

4.2 Methods for Identifying or Evaluating Substances/Compounds

The methods described herein are designed to identify substances that modulate the biological activity of a tumor associated protein (e.g. BTA Related Protein) including substances that bind to tumor associated proteins, or bind to other proteins that interact with a tumor associated protein, to compounds that interfere with, or enhance the interaction of a tumor associated protein and substances that bind to a tumor associated protein or other proteins that interact with a tumor associated protein. Methods are also utilized that identify compounds that bind to tumor associated protein nucleic acid regulatory sequences.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, carbohydrates, oligosaccharides, polysaccharides, and Fab expression library fragments, and epitope-binding fragments thereof)], and small organic or inorganic molecules. The substance or compound may be an endogenous physiological compound or it may be a natural or synthetic compound.

Substances which modulate a tumor associated protein (e.g. BTA Related Protein) can be identified based on their ability to bind to a tumor associated protein (e.g BTA Related Protein). Therefore, the invention also provides methods for identifying substances which bind to a tumor associated protein (e.g. BTA Related Protein). Substances identified using the methods of the invention may be isolated, cloned and sequenced using conventional techniques. A substance that associates with a tumor associated protein (e.g. BTA Related Protein) may be an agonist or antagonist of the biological or immunological activity of a tumor associated protein.

The term “agonist” refers to a molecule that increases the amount of, or prolongs the duration of, the activity of the polypeptide. The term “antagonist” refers to a molecule which decreases the biological or immunological activity of the polypeptide. Agonists and antagonists may include proteins, nucleic acids, carbohydrates, or any other molecules that associate with a polypeptide of the invention.

Substances which can bind with a tumor associated protein (e.g. BTA Related Protein) may be identified by reacting a tumor associated protein with a test substance which potentially binds to a tumor associated protein, under conditions which permit the formation of substance-tumor associated protein complexes and removing and/or detecting the complexes. The complexes can be detected by assaying for substance-tumor associated protein complexes, for free substance, or for non-complexed tumor associated protein. Conditions which permit the formation of substance-tumor associated protein complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the tumor associated protein or the substance, or labeled tumor associated protein, or a labeled substance may be utilized. The antibodies, proteins, or substances may be labeled with a detectable substance as described above.

A tumor associated protein (e.g. BTA Related Protein), or the substance used in the method of the invention may be insolubilized. For example, a tumor associated protein, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling.

The invention also contemplates a method for evaluating a compound for its ability to modulate the biological activity of a tumor associated protein (e.g. BTA Related Protein), by assaying for an agonist or antagonist (i.e. enhancer or inhibitor) of the binding of a tumor associated protein with a substance which binds with a tumor associated protein. The basic method for evaluating if a compound is an agonist or antagonist of the binding of a tumor associated protein and a substance that binds to the protein, is to prepare a reaction mixture containing the tumor associated protein and the substance under conditions which permit the formation of substance-tumor associated protein complexes, in the presence of a test compound. The test compound may be initially added to the mixture, or may be added subsequent to the addition of the tumor associated protein and substance. Control reaction mixtures without the test compound or with a placebo are also prepared. The formation of complexes is detected and the formation of complexes in the control reaction but not in the reaction mixture indicates that the test compound interferes with the interaction of the tumor associated protein and substance. The reactions may be carried out in the liquid phase or the tumor associated protein, substance, or test compound may be immobilized as described herein. The ability of a compound to modulate the biological activity of a tumor associated protein of the invention may be tested by determining the biological effects on cells.

It will be understood that the agonists and antagonists i.e. inhibitors and enhancers that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that inhibit the effects of an agonist of the interaction of a tumor associated protein (e.g. BTA Related Protein) with a substance which is capable of binding to the tumor associated protein. Thus, the invention may be used to assay for a compound that competes for the same binding site of a tumor associated protein.

The invention also contemplates methods for identifying compounds that bind to proteins that interact with a tumor associated protein (e.g. BTA Related Protein). Protein-protein interactions may be identified using conventional methods such as co-immunoprecipitation, crosslinking and co-purification through gradients or chromatographic columns. Methods may also be employed that result in the simultaneous identification of genes which encode proteins interacting with a tumor associated protein (e.g. BTA Related Protein). These methods include probing expression libraries with labeled tumor associated proteins.

Two-hybrid systems may also be used to detect protein interactions in vivo. Generally, plasmids are constructed that encode two hybrid proteins. A first hybrid protein consists of the DNA-binding domain of a transcription activator protein fused to a tumor associated protein, and the second hybrid protein consists of the transcription activator protein's activator domain fused to an unknown protein encoded by a cDNA which has been recombined into the plasmid as part of a cDNA library. The plasmids are transformed into a strain of yeast (e.g. S. cerevisiae) that contains a reporter gene (e.g. lacZ, luciferase, alkaline phosphatase, horseradish peroxidase) whose regulatory region contains the transcription activator's binding site. The hybrid proteins alone cannot activate the transcription of the reporter gene. However, interaction of the two hybrid proteins reconstitutes the functional activator protein and results in expression of the reporter gene, which is detected by an assay for the reporter gene product.

It will be appreciated that fusion proteins may be used in the above-described methods. In particular, tumor associated proteins (e.g. BTA Related Proteins) fused to a glutathione-S-transferase may be used in the methods.

The reagents suitable for applying the methods of the invention to evaluate compounds that modulate a tumor associated protein (e.g. BTA Related Protein) may be packaged into convenient kits providing the necessary materials packaged into suitable containers. The kits may also include suitable supports useful in performing the methods of the invention.

4.3 Compositions and Treatments

The tumor associated proteins of the invention, substances or compounds that modulate tumor associated proteins, antibodies, and nucleic acid molecules encoding tumor associated proteins may be used for modulating the biological activity of a tumor associated protein described herein, and they may be used in the treatment of conditions such as cancer (particularly breast cancer). In particular, the proteins, antibodies, substances, compounds, and nucleic acid molecules, may be used to treat patients who have a tumor associated protein in, or on, their cancer cells. In an embodiment, the tumor associated protein is a BTA Related Protein.

Accordingly, the proteins, substances, antibodies, and compounds may be formulated into pharmaceutical compositions for administration to subjects in a biologically compatible form suitable for administration in vivo. By “biologically compatible form suitable for administration in vivo” is meant a form of the active substance to be administered in which any toxic effects are outweighed by the therapeutic effects. The active substances may be administered to patients including humans, and animals. Administration of a therapeutically active amount of a pharmaceutical composition of the present invention is defined as an amount effective, at dosages and for periods of time necessary to achieve the desired result. For example, a therapeutically active amount of a substance may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). For example, the compositions may be used in combination with anti-proliferative agents, antimicrobial agents, immunostimulatory agents, or anti-inflammatories. In particular, the compounds may be used in combination with anti-viral and/or anti-proliferative agents. The compositions of the invention may be administered concurrently, separately, or sequentially with other therapeutic agents or therapies.

Vectors derived from retroviruses, adenovirus, herpes or vaccinia viruses, or from various bacterial plasmids, may be used to deliver nucleic acid molecules encoding a tumor associated protein to a targeted organ, tissue, or cell population. Methods well known to those skilled in the art may be used to construct recombinant vectors which will express antisense nucleic acid molecules. (See, for example, the techniques described in Sambrook et al (supra) and Ausubel et al (supra)).

The nucleic acid molecules comprising full length cDNA sequences and/or their regulatory elements enable a skilled artisan to use sequences encoding a tumor associated protein as an investigative tool in sense (Youssoufian H and H F Lodish 1993 Mol Cell Biol 13:98-104) or antisense (Eguchi et al (1991) Annu Rev Biochem 60:631-652) regulation of gene function. Such technology is well known in the art, and sense or antisense oligomers, or larger fragments, can be designed from various locations along the coding or control regions.

Genes encoding a tumor associated protein can be turned off by transfecting a cell or tissue with vectors which express high levels of a desired tumor associated protein encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a tumor associated protein, i.e. the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a tumor associated protein.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

An antibody against a tumor associated protein may be conjugated to chemotherapeutic drugs, toxins, immunological response modifiers, hematogenous agents, enzymes, and radioisotopes and used in the prevention and treatment of cancer (e.g. breast cancer). For example, an antibody against a tumor associated protein may be conjugated to toxic moieties including but not limited to ricin A, diphtheria toxin, abrin, modeccin, or bacterial toxins from Pseudomonas or Shigella. Toxins and their derivatives have been reported to form conjugates with antibodies specific to particular target tissues, such as cancer or tumor cells in order to obtain specifically targeted cellular toxicity (Moolten F. L. et al, Immun. Rev. 62:47-72, 1982, and Bernhard, M. I. Cancer Res. 43:4420, 1983).

Conjugates can be prepared by standard means known in the art. A number of bifunctional linking agents (e.g. heterobifunctional linkers such as N-succinimidyl-3-(2-pyridyldithio)propionate) are available commercially from Pierce Chemically Company, Rockford, Ill.

Administration of the antibodies or immunotoxins for therapeutic use may be by an intravenous route, although with proper formulation additional routes of administration such as intraperitoneal, oral, or transdermal administration may also be used.

A tumor associated protein may be conjugated to chemotherapeutic drugs, toxins, immunological response modifiers, enzymes, and radioisotopes using methods known in the art.

The invention also provides immunotherapeutic approaches for preventing or reducing the severity of a cancer. The clinical signs or symptoms of the cancer in a subject are indicative of a beneficial effect to the patient due to the stimulation of the subject's immune response against the cancer. Stimulating an immune response refers to inducing an immune response or enhancing the activity of immunoeffector cells in response to administration of a vaccine preparation of the invention. The prevention of a cancer can be indicated by an increased time before the appearance of cancer in a patient that is predisposed to developing cancer due for example to a genetic disposition or exposure to a carcinogenic agent. The reduction in the severity of a cancer can be indicated by a decrease in size or growth rate of a tumor.

Vaccines can be derived from a tumor associated protein (e.g. BTA Related Protein), peptides derived therefrom, or chemically produced synthetic peptides, or any combination of these molecules, or fusion proteins or peptides thereof. The proteins, peptides, etc. can be synthesized or prepared recombinantly or otherwise biologically, to comprise one or more amino acid sequences corresponding to one or more epitopes of a tumor associated protein.

Epitopes of a tumor associated protein will be understood to include the possibility that in some instances amino acid sequence variations of a naturally occurring protein or polypeptide may be antigenic and confer protective immunity against cancer or anti-tumorigenic effects. Sequence variations may include without limitation, amino acid substitutions, extensions, deletions, truncations, interpolations, and combinations thereof. Such variations fall within the scope of the invention provided the protein containing them is immunogenic and antibodies against such polypeptide cross-react with naturally occurring tumor associated proteins to a sufficient extent to provide protective immunity and/or anti-tumorigenic activity when administered as a vaccine.

The proteins, peptides etc, can be incorporated into vaccines capable of inducing an immune response using methods known in the art. Nucleic acids encoding immunogenic proteins or peptides have been shown to be useful in this regard. Viral vectors that have been successfully utilized for introducing a nucleic acid to a host include retrovirus, adenovirus, adeno-associated virus (AAV), herpes virus, and poxvirus, among others. It is understood in the art that many such viral vectors are available in the art. The vectors of the present invention may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), and PCR Protocols: A Guide to Methods and Applications (Innis, et al. 1990. Academic Press, San Diego, Calif.).

Preferred retroviral vectors are derivatives of lentivirus as well as derivatives of murine or avian retroviruses. Examples of suitable retroviral vectors include, for example, Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of retroviral vectors can incorporate multiple exogenous nucleic acid sequences. As recombinant retroviruses are defective, they require assistance in order to produce infectious vector particles. This assistance can be provided by, for example, helper cell lines encoding retrovirus structural genes. Suitable helper cell lines include ψ2, PA317 and PA12, among others. The vector virions produced using such cell lines may then be used to infect a tissue cell line, such as NIH 3T3 cells, to produce large quantities of chimeric retroviral virions. Retroviral vectors may be administered by traditional methods (i.e., injection) or by implantation of a “producer cell line” in proximity to the target cell population (Culver, K., et al., 1994, Hum. Gene Ther., 5 (3): 343-79; Culver, K., et al., Cold Spring Harb. Symp. Quant. Biol., 59: 685-90); Oldfield, E., 1993, Hum. Gene Ther., 4 (1): 39-69). The producer cell line is engineered to produce a viral vector and releases viral particles in the vicinity of the target cell. A portion of the released viral particles contact the target cells and infect those cells, thus delivering a nucleic acid of the present invention to the target cell. Following infection of the target cell, expression of the nucleic acid of the vector occurs.

Adenoviral vectors have proven especially useful for gene transfer into eukaryotic cells (Rosenfeld, M., et al., 1991, Science, 252 (5004): 431-4; Crystal, R., et al., 1994, Nat. Genet., 8 (1): 42-51), the study eukaryotic gene expression (Levrero, M., et al., 1991, Gene, 101 (2): 195-202), vaccine development (Graham, F. and Prevec, L., 1992, Biotechnology, 20: 363-90), and in animal models (Stratford-Perricaudet, L., et al., 1992, Bone Marrow Transplant., 9 (Suppl. 1): 151-2; Rich, D., et al., 1993, Hum. Gene Ther., 4 (4): 461-76). Experimental routes for administrating recombinant Ad to different tissues in vivo have included intratracheal instillation (Rosenfeld, M., et al., 1992, Cell, 68 (1): 143-55) injection into muscle (Quantin, B., et al., 1992, Proc. Natl. Acad. Sci. U.S.A., 89 (7): 2581-4), peripheral intravenous injection (Herz, J., and Gerard, R., 1993, Proc. Natl. Acad. Sci. U.S.A., 90 (7): 2812-6) and stereotactic inoculation to brain (Le Gal La Salle, G., et al., 1993, Science, 259 (5097): 988-90), among others.

Adeno-associated virus (AAV) demonstrates high-level infectivity, broad host range and specificity in integrating into the host cell genome (Hermonat, P., et al., 1984, Proc. Natl. Acad. Sci. U.S.A., 81 (20): 6466-70). And Herpes Simplex Virus type-1 (HSV-1) is yet another attractive vector system, especially for use in the nervous system because of its neurotropic property (Geller, A., et al., 1991, Trends Neurosci., 14 (10): 428-32; Glorioso, et al., 1995, Mol. Biotechnol., 4 (1): 87-99; Glorioso, et al., 1995, Annu. Rev. Microbiol., 49: 675-710).

Poxvirus is another useful expression vector (Smith, et al. 1983, Gene, 25 (1): 21-8; Moss, et al, 1992, Biotechnology, 20: 345-62; Moss, et al, 1992, Curr. Top. Microbiol. Immunol., 158: 25-38; Moss, et al. 1991. Science, 252: 1662-1667). Poxviruses shown to be useful include vaccinia, NYVAC, avipox, fowlpox, canarypox, ALVAC, and ALVAC(2), among others.

NYVAC (vP866) was derived from the Copenhagen vaccine strain of vaccinia virus by deleting six nonessential regions of the genome encoding known or potential virulence factors (see, for example, U.S. Pat. Nos. 5,364,773 and 5,494,807). The deletion loci were also engineered as recipient loci for the insertion of foreign genes. The deleted regions are: thymidine kinase gene (TK; J2R); hemorrhagic region (u; B13R+B14R); A type inclusion body region (ATI; A26L); hemagglutinin gene (HA; A56R); host range gene region (C7L-K1L); and, large subunit, ribonucleotide reductase (I4L). NYVAC is a genetically engineered vaccinia virus strain that was generated by the specific deletion of eighteen open reading frames encoding gene products associated with virulence and host range. NYVAC has been show to be useful for expressing TAs (see, for example, U.S. Pat. No. 6,265,189). NYVAC (vP866), vP994, vCP205, vCP1433, placZH6H4Lreverse, pMPC6H6K3E3 and pC3H6FHVB were also deposited with the ATCC under the terms of the Budapest Treaty, accession numbers VR-2559, VR-2558, VR-2557, VR-2556, ATCC-97913, ATCC-97912, and ATCC-97914, respectively.

ALVAC-based recombinant viruses (i.e., ALVAC-1 and ALVAC-2) are also suitable for use in practicing the present invention (see, for example, U.S. Pat. No. 5,756,103). ALVAC(2) is identical to ALVAC(1) except that ALVAC(2) genome comprises the vaccinia E3L and K3L genes under the control of vaccinia promoters (U.S. Pat. No. 6,130,066; Beattie et al., 1995a, 1995b, 1991; Chang et al., 1992; Davies et al., 1993). Both ALVAC(1) and ALVAC(2) have been demonstrated to be useful in expressing foreign DNA sequences, such as TAs (Tartaglia et al., 1993 a,b; U.S. Pat. No. 5,833,975). ALVAC was deposited under the terms of the Budapest Treaty with the American Type Culture Collection (ATCC), 10801 University Boulevard, Manassas, Va. 20110-2209, USA, ATCC accession number VR-2547.

Another useful poxvirus vector is TROVAC. TROVAC refers to an attenuated fowlpox that was a plaque-cloned isolate derived from the FP-1 vaccine strain of fowlpoxvirus which is licensed for vaccination of 1-day old chicks. TROVAC was likewise deposited under the terms of the Budapest Treaty with the ATCC, accession number 2553.

“Non-viral” plasmid vectors may also be suitable in practicing the present invention. Preferred plasmid vectors are compatible with bacterial, insect, and/or mammalian host cells. Such vectors include, for example, PCR-II, pCR3, and pcDNA3.1 (Invitrogen, San Diego, Calif.), pBSII (Stratagene, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII, Invitrogen), pDSR-alpha (PCT pub. No. WO 90/14363) and pFastBacDual (Gibco-BRL, Grand Island, N.Y.) as well as Bluescript™ plasmid derivatives (a high copy number COLE1-based phagemid, Stratagene Cloning Systems, La Jolla, Calif.), PCR cloning plasmids designed for cloning Taq-amplified PCR products (e.g., TOPO™ TA cloning® kit, PCR2.1® plasmid derivatives, Invitrogen, Carlsbad, Calif.). Bacterial vectors may also be used with the current invention. These vectors include, for example, Shigella, Salmonella, Vibrio cholerae, Lactobacillus, Bacille calmette guérin (BCG), and Streptococcus (see for example, WO 88/6626; WO 90/0594; WO 91/13157; WO 92/1796; and WO 92/21376). Many other non-viral plasmid expression vectors and systems are known in the art and could be used with the current invention.

Suitable nucleic acid delivery techniques include DNA-ligand complexes, adenovirus-ligand-DNA complexes, direct injection of DNA, CaPO₄ precipitation, gene gun techniques, electroporation, and colloidal dispersion systems, among others. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6: 77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Techniques for enhancing the antigenicity of the proteins, peptides, etc. are known in the art and include incorporation into a multimeric structure, binding to a highly immunogenic protein carrier, for example, keyhole limpet hemocyanin (KLH), or diptheria toxoid, and administration in combination with adjuvants or any other enhancer of immune response.

Vaccines may be combined with physiologically acceptable media, including immunologically acceptable diluents and carriers as well as commonly employed adjuvants such as Freund's Complete Adjuvant, saponin, alum, and the like. Other suitable adjuvants are shown in Table 1.

It will be further appreciated that anti-idiotype antibodies to antibodies to tumor associated proteins described herein are also useful as vaccines and can be similarly formulated.

The administration of a vaccine in accordance with the invention, is generally applicable to the prevention or treatment of cancers including breast cancer.

The administration to a patient of a vaccine in accordance with the invention for the prevention and/or treatment of cancer can take place before or after a surgical procedure to remove the cancer, before or after a chemotherapeutic procedure for the treatment of cancer, and before or after radiation therapy for the treatment of cancer and any combination thereof. The cancer immunotherapy in accordance with the invention would be a preferred treatment for the prevention and/or treatment of cancer, since the side effects involved are substantially minimal compared with the other available treatments e.g. surgery, chemotherapy, radiation therapy. The vaccines have the potential or capability to prevent cancer in subjects without cancer but who are at risk of developing cancer.

The activity of the proteins, substances, compounds, antibodies, nucleic acid molecules, agents, and compositions of the invention may be confirmed in animal experimental model systems. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

4.4 Other Applications

Nucleic acid molecules encoding BTA Related Proteins may be used in molecular biology techniques that have not yet been developed, provided the new techniques rely on properties of nucleotide sequences that are currently known, including but not limited to such properties as the triplet genetic code and specific base pair interactions.

The invention also provides methods for studying the function of a nucleic acid or protein of the invention. Cells, tissues, and non-human animals lacking in expression or partially lacking in expression of a nucleic acid molecule or gene encoding a protein ot the invention (e.g. BTA Related Protein) may be developed using recombinant expression vectors of the invention having specific deletion or insertion mutations in the gene. A recombinant expression vector may be used to inactivate or alter the endogenous gene by homologous recombination, and thereby create a deficient cell, tissue, or animal.

Null alleles may be generated in cells, such as embryonic stem cells by deletion mutation. A recombinant gene may also be engineered to contain an insertion mutation that inactivates the gene. Such a construct may then be introduced into a cell, such as an embryonic stem cell, by a technique such as transfection, electroporation, injection etc. Cells lacking an intact gene may then be identified, for example by Southern blotting, Northern Blotting, or by assaying for expression of the encoded polypeptide using the methods described herein. Such cells may then be fused to embryonic stem cells to generate transgenic non-human animals deficient in a BTA Related Protein. Germline transmission of the mutation may be achieved, for example, by aggregating the embryonic stem cells with early stage embryos, such as 8 cell embryos, in vitro; transferring the resulting blastocysts into recipient females and; generating germline transmission of the resulting aggregation chimeras. Such a mutant animal may be used to define specific cell populations, developmental patterns and in vivo processes, normally dependent on gene expression.

The invention thus provides a transgenic non-human mammal all of whose germ cells and somatic cells contain a recombinant expression vector that inactivates or alters a nucleic acid molecule of the invention. In an embodiment the invention provides a transgenic non-human mammal all of whose germ cells and somatic cells contain a recombinant expression vector that inactivates or alters a gene or a nucleic acid molecule of the invention resulting in a BTA Related Protein associated pathology. Further the invention provides a transgenic non-human mammal which does not express or has altered expression of a nucleic acid molecule or BTA Related Protein of the invention. In an embodiment, the invention provides a transgenic non-human mammal which does not express or has altered (e.g. reduced) expression of a nucleic acid or protein of the invention resulting in a BTA Related Protein associated pathology. A BTA Related Protein associated pathology refers to a phenotype observed for a homozygous or heterozygous mutant.

A transgenic non-human animal includes but is not limited to mouse, rat, rabbit, sheep, hamster, dog, cat, goat, and monkey, preferably mouse.

The invention also provides a transgenic non-human animal assay system which provides a model system for testing for an agent that reduces or inhibits a pathology associated with a nucleic acid molecule or a protein of the invention, preferably a BTA Related Protein associated pathology, comprising:

(a) administering the agent to a transgenic non-human animal of the invention; and

(b) determining whether said agent reduces or inhibits the pathology (e.g. BTA Related Protein associated pathology) in the transgenic non-human animal relative to a transgenic non-human animal of step (a) which has not been administered the agent.

The agent may be useful in the treatment and prophylaxis of conditions such as cancer as discussed herein. The agents may also be incorporated in pharmaceutical compositions as described herein.

The following non-limiting examples are illustrative of the present invention:

### EXAMPLE 1

Immunoscreening of a cDNA expression library with breast cancer serum was conducted. In this study, the Uni-ZAP™ pre-made breast carcinoma cDNA expression library purchased from Stratagene was used. Breast cancer serum was obtained from a patient with highly metastatic breast cancer. The details of this screening method are as follows: the cDNA library was plated on NZY agar plates at a density of 500 clones/15 cm plate. The plates were incubated at 42° C. for 6 hours to allow plaques to develop. Nitrocellulose filters soaked with IPTG were then laid on top of the plaques and incubated at 37° C. overnight to induce the expression of the proteins and to transfer the plaques onto the membranes. The filters were then blocked with 5% non-fat dried milk/PBS-T at room temperature for 2 hours.

To screen the library, the serum was diluted 1000-fold in 5% non-fat dried milk/PBS-T. The diluted serum was first incubated with E. coli phage lysate (purchased from Stratagene) for 2 hours at room temperature to minimize the cross-reaction between autoantibodies and the bacterial, phage proteins. Nitrocellulose filters were incubated with this pre-absorbed serum overnight at 4° C. to identify the cellular proteins that react with the autoantibodies in serum. Following probing with serum, the filters were further treated with goat anti-human IgG conjugated with alkaline phosphatase (Jackson Immunoresearch) diluted 2000-fold in 5% non-fat dried milk/PBS-T for 1 hour at room temperature, then proceeded to chemiluminescence detection with dioxetane based-substrate (AMPPD).

The plaques exhibiting immunoreactivity were excised from the plates and the phages were eluted into SM buffer. These phages were then converted to the pBluescript phagemid form by in vivo excision with Exassist™ helper phage following the manufacturer's instructions. The excised phagemids were purified and subjected to automated DNA sequencing with M13 forward and reverse primers. The insert sequences were compared to the known sequences in Genbank database with the BLAST alignment program.

With this method, about 1×10⁴ clones were screened. Seven distinct clones were found to have immunoreactivities with breast cancer serum. These clones were excised and sequenced. When these sequences were compared to the Genbank database, five of them were matched with sequences encoding for known proteins. For the other two sequences, one encodes for a novel protein, the other sequence matches with a genomic sequence on chromosome 7q31, but it does not encode for any protein sequence deposited in Genbank (Table 2).

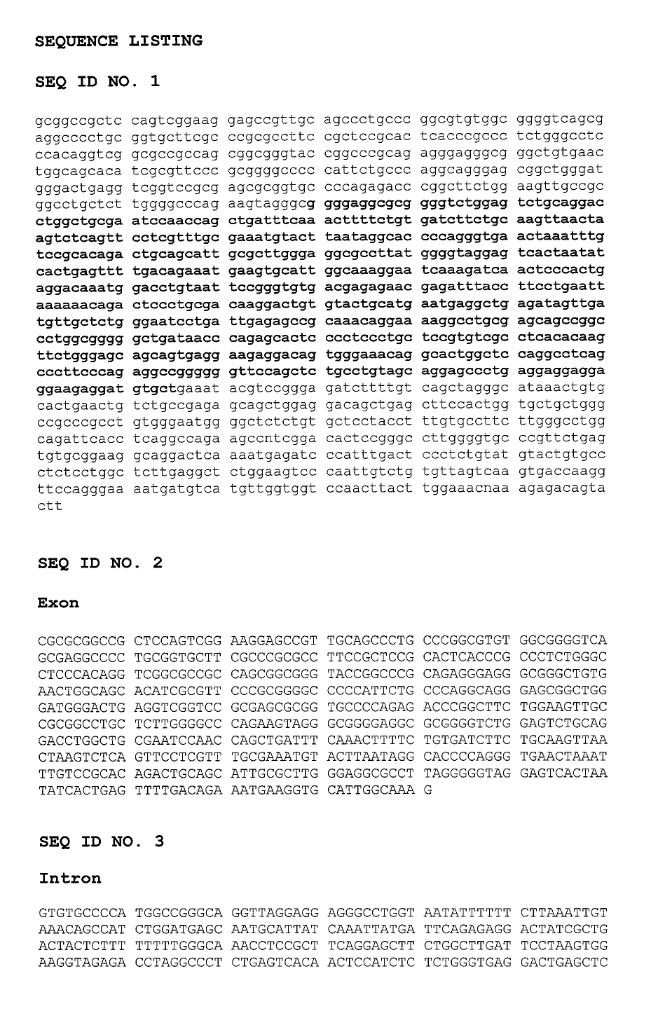

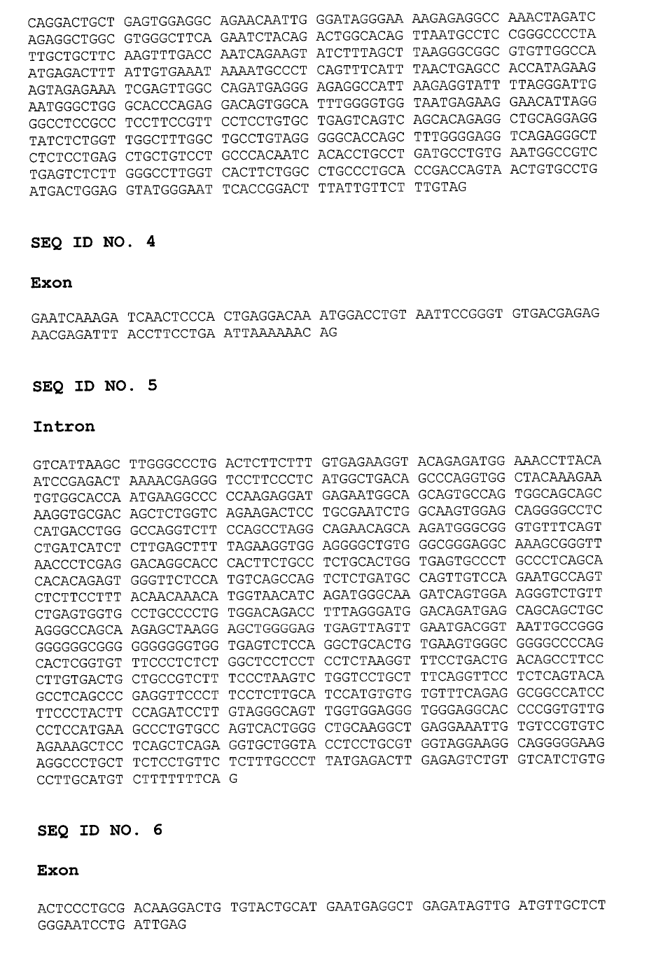

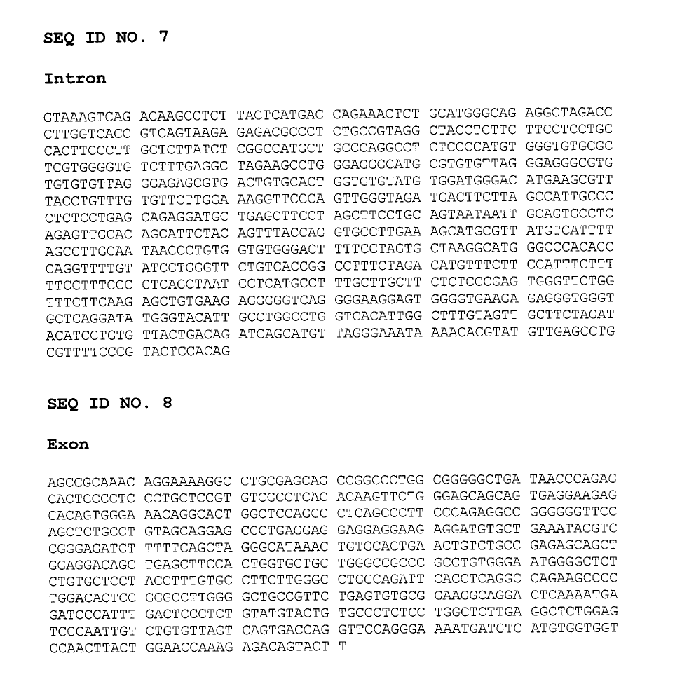

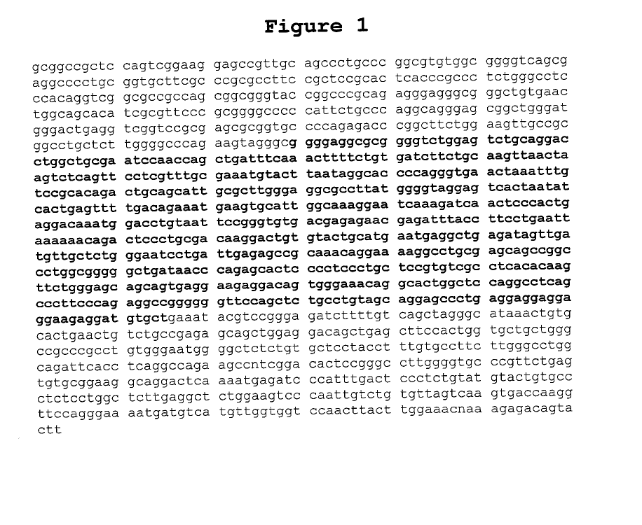

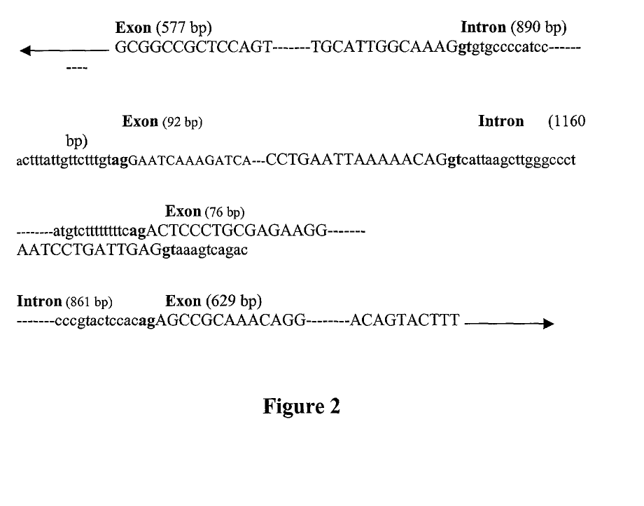

The new sequence encodes a novel gene. The total length of this sequence identified by immunoscreening is about 660 bp. When this sequence was used to blast against the human EST database, it matches with a number of ESTs. With ESTs AW975482, AW410740, and N73248, and a continuous sequence of 1.4 kb was constructed (FIG. 1). When this constructed sequence was compared to the genomic sequence on chromosome 7q31 (AC009542), four exons with 3 intervening introns were identified. The intron-exon boundaries are completely conserved (“GT” is in the splice donor site and the “AG” is in the splice acceptor site) (FIG. 2). No poly A tail at the 3′ end or Kozak consensus sequence for translation initiation at the 5′ end was identified in the constructed sequence. When this DNA sequence was translated into protein sequence with combination of three reading frames, some significant amino acid stretches were found.

In summary, cellular components which trigger an immune response in breast cancer were identified by immunoscreening a breast cDNA expression library with breast cancer serum. Nine proteins that appear to exhibit immunoreactivities were identified (Table 2).

TABLE 1
TYPES OF IMMUNOLOGIC ADJUVANTS

| | TYPE OF | | |
|---|---|---|---|
| | ADJUVANT | GENERAL EXAMPLES | SPECIFIC EXAMPLES/REFERENCES |
| 1 | GEL-TYPE | ALUMINUM | (AGGERBECK AND HERON, 1995) |
| | | HYDROXIDE/PHOSPHATE (“ALUM | |
| | | ADJUVANTS”) | |
| | | CALCIUM PHOSPHATE | (RELYVELD, 1986) |
| 2 | MICROBIAL | MURAMYL DIPEPTIDE (MDP) | (CHEDID ET AL., 1986) |
| | | BACTERIAL EXOTOXINS | CHOLERA TOXIN (CT), E. COLI |
| | | | LABILE TOXIN (LT)(FREYTAG AND |
| | | | CLEMENTS, 1999) |
| | | ENDOTOXIN-BASED ADJUVANTS | MONOPHOSPHORYL LIPID A (MPL) |
| | | | (ULRICH AND MYERS, 1995) |
| | | OTHER BACTERIAL | CPG OLIGONUCLEOTIDES |
| | | | (CORRAL AND PETRAY, 2000), BCG |
| | | | SEQUENCES (KRIEG, ET AL. |
| | | | NATURE, 374: 576), TETANUS |
| | | | TOXOID (RICE, ET AL. J. IMMUNOL., |
| | | | 2001, 167: 1558-1565) |
| 3 | PARTICULATE | BIODEGRADABLE | (GUPTA ET AL., 1998) |
| | | POLYMER MICROSPHERES | |
| | | IMMUNOSTIMULATORY | (MOREIN AND BENGTSSON, 1999) |
| | | COMPLEXES (ISCOMS) | |
| | | LIPOSOMES | (WASSEF ET AL., 1994) |
| 4 | OIL- | FREUND'S INCOMPLETE | (JENSEN ET AL., 1998) |
| | EMULSION | ADJUVANT | |
| | AND | | |
| | SURFACTANT- | | |
| | BASED | | |
| | ADJUVANTS | | |
| | | MICROFLUIDIZED EMULSIONS | MF59 (OTT ET AL., 1995) |
| | | | SAF (ALLISON AND BYARS, 1992) |
| | | | (ALLISON, 1999) |
| | | SAPONINS | QS-21 (KENSIL, 1996) |
| 5 | SYNTHETIC | MURAMYL PEPTIDE DERIVATIVES | MURABUTIDE (LEDERER, 1986) |
| | | | THREONY-MDP (ALLISON, 1997) |
| | | NONIONIC BLOCK COPOLYMERS | L121 (ALLISON, 1999) |
| | | POLYPHOSPHAZENE (PCPP) | (PAYNE ET AL., 1995) |
| | | SYNTHETIC POLYNUCLEOTIDES | POLY A: U, POLY I: C (JOHNSON, |
| | | | 1994) |

TABLE 2
Proteins identified by the immunoscreening of a breast carcinoma cDNA expression library
with breast cancer serum.

| Clone name | Identity (Genbank Accession number) | Function |
|---|---|---|
| 607-1 | Protein phosphatase 4 regulatory subunit | Microtubule organization at |
| | (AJ271448) | centrosome |
| 607-2 | Unnamed protein product (hypothetical protein, | Unknown |
| | AK001674) | |
| 608-1♦ | Unknown, match with genomic sequence from | Unknown |
| | chromosome 7q31 (AC009542) | |
| 608-2 | Putative translation initiation factor (SUI1) (NM- | Protein translation |
| | 005801) | |
| 608-3 | Hypothetical RNA helicase (AL359945) | Transcription |
| 608-4 | MIL1 protein, nuclear gene encoding | Promote cell survival |
| | mitochondrial protein (NM-015367) | |
| 623-1 | MacMarks protein (X70326) | Signal transduction |
| 629-1 | Chromosome 11 open reading frame 10 (c11orf10) | Unknown |
| | (AF086763) | |
| 608-6 | Plakophilin 4 (PKP4) (NM_003628) | Maintenance of cellular |
| | | structure and cell contacts |

(♦This is the novel gene identified.)

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

| | |
|---|---|
| gcggccgctc cagtcggaag gagccgttgc agccctgccc ggcgtgtggc ggggtcagcg | SEQ ID NO.1 |
| aggcccctgc ggtgcttcgc ccgcgccttc cgctccgcac tcacccgccc tctgggcctc | |
| ccacaggtcg gcgccgccag cggcgggtac cggcccgcag agggagggcg ggctgtgaac | |
| tggcagcaca tcgcgttccc gcggggcccc cattctgccc aggcagggag cggctgggat | |
| gggactgagg tcggtccgcg agcgcggtgc cccagagacc cggcttctgg aagttgccgc | |
| ggcctgctct tggggcccag aagtagggcg gggaggcgcg gggtctggag tctgcaggac | |
| ctggctgcga atccaaccag ctgatttcaa acttttctgt gatcttctgc aagttaacta | |
| agtctcagtt cctcgtttgc gaaatgtact taataggcac cccagggtga actaaatttg | |
| tccgcacaga ctgcagcatt gcgcttggga ggcgccttat ggggtaggag tcactaatat | |
| cactgagttt tgacagaaat gaagtgcatt ggcaaaggaa tcaaagatca actcccactg | |
| aggacaaatg gacctgtaat tccgggtgtg acgagagaac gagatttacc ttcctgaatt | |
| aaaaaacaga ctccctgcga caaggactgt gtactgcatg aatgaggctg agatagttga | |
| tgttgctctg ggaatcctga ttgagagccg caaacaggaa aaggcctgcg agcagccggc | |
| cctggcgggg gctgataacc cagagcactc ccctccctgc tccgtgtcgc ctcacacaag | |
| ttctgggagc agcagtgagg aagaggacag tgggaaacag gcactggctc caggcctcag | |
| cccttcccag aggccggggg gttccagctc tgcctgtagc aggagccctg aggaggagga | |
| ggaagaggat gtgctgaaat acgtccggga gatcttttgt cagctagggc ataaactgtg | |
| cactgaactg tctgccgaga gcagctggag gacagctgag cttccactgg tgctgctggg | |
| ccgcccgcct gtgggaatgg ggctctctgt gctcctacct ttgtgccttc ttgggcctgg | |
| cagattcacc tcaggccaga agccntcgga cactccgggc cttggggtgc ccgttctgag | |
| tgtgcggaag gcaggactca aaatgagatc ccatttgact ccctctgtat gtactgtgcc | |
| ctctcctggc tcttgaggct ctggaagtcc caattgtctg tgttagtcaa gtgaccaagg | |
| ttccagggaa aatgatgtca tgttggtggt ccaacttact tggaaacnaa agagacagta | |
| ctt | |

Exon

| | |
|---|---|
| CGCGCGGCCG CTCCAGTCGG AAGGAGCCGT TGCAGCCCTG CCCGGCGTGT GGCGGGGTCA | SEQ ID NO.2 |
| GCGAGGCCCC TGCGGTGCTT CGCCCGCGCC TTCCGCTCCG CACTCACCCG CCCTCTGGGC | |
| CTCCCACAGG TCGGCGCCGC CAGCGGCGGG TACCGGCCCG CAGAGGGAGG GCGGGCTGTG | |
| AACTGGCAGC ACATCGCGTT CCCGCGGGGC CCCCATTCTG CCCAGGCAGG GAGCGGCTGG | |
| GATGGGACTG AGGTCGGTCC GCGAGCGCGG TGCCCCAGAG ACCCGGCTTC TGGAAGTTGC | |
| CGCGGCCTGC TCTTGGGGCC CAGAAGTAGG GCGGGGAGGC GCGGGGTCTG GAGTCTGCAG | |
| GACCTGGCTG CGAATCCAAC CAGCTGATTT CAAACTTTTC TGTGATCTTC TGCAAGTTAA | |
| CTAAGTCTCA GTTCCTCGTT TGCGAAATGT ACTTAATAGG CACCCCAGGG TGAACTAAAT | |
| TTGTCCGCAC AGACTGCAGC ATTGCGCTTG GGAGGCGCCT TAGGGGGTAG GAGTCACTAA | |
| TATCACTGAG TTTTGACAGA AATGAAGGTG CATTGGCAAA G | |

Intron

| | |
|---|---|
| GTGTGCCCCA TGGCCGGGCA GGTTAGGAGG AGGGCCTGGT AATATTTTTT CTTAAATTGT | SEQ ID NO.3 |
| AAACAGCCAT CTGGATGAGC AATGCATTAT CAAATTATGA TTCAGAGAGG ACTATCGCTG | |
| ACTACTCTTT TTTTTGGGCA AACCTCCGCT TCAGGAGCTT CTGGCTTGAT TCCTAAGTGG | |
| AAGGTAGAGA CCTAGGCCCT CTGAGTCACA ACTCCATCTC TCTGGGTGAG GACTGAGCTC | |
| CAGGACTGCT GAGTGGAGGC AGAACAATTG GGATAGGGAA AAGAGAGGCC AAACTAGATC | |
| AGAGGCTGGC GTGGGCTTCA GAATCTACAG ACTGGCACAG TTAATGCCTC CGGGCCCCTA | |
| TTGCTGCTTC AAGTTTGACC AATCAGAAGT ATCTTTAGCT TAAGGGCGGC GTGTTGGCCA | |
| ATGAGACTTT ATTGTGAAAT AAAATGCCCT CAGTTTCATT TAACTGAGCC ACCATAGAAG | |
| AGTAGAGAAA TCGAGTTGGC CAGATGAGGG AGAGGCCATT AAGAGGTATT TTAGGGATTG | |
| AATGGGCTGG GCACCCAGAG GACAGTGGCA TTTGGGGTGG TAATGAGAAG GAACATTAGG | |
| GGCCTCCGCC TCCTTCCGTT CCTCCTGTGC TGAGTCAGTC AGCACAGAGG CTGCAGGAGG | |
| TATCTCTGGT TGGCTTTGGC TGCCTGTAGG GGGCACCAGC TTTGGGGAGG TCAGAGGGCT | |
| CTCTCCTGAG CTGCTGTCCT GCCCACAATC ACACCTGCCT GATGCCTGTG AATGGCCGTC | |
| TGAGTCTCTT GGGCCTTGGT CACTTCTGGC CTGCCCTGCA CCGACCAGTA ACTGTGCCTG | |
| ATGACTGGAG GTATGGGAAT TCACCGGACT TTATTGTTCT TTGTAG | |

Exon

| | |
|---|---|
| GAATCAAAGA TCAACTCCCA CTGAGGACAA ATGGACCTGT AATTCCGGGT GTGACGAGAG | SEQ ID NO.4 |
| AACGAGATTT ACCTTCCTGA ATTAAAAAAC AG | |

Intron

| | |
|---|---|
| GTCATTAAGC TTGGGCCCTG ACTCTTCTTT GTGAGAAGGT ACAGAGATGG AAACCTTACA | SEQ ID NO.5 |
| ATCCGAGACT AAAACGAGGG TCCTTCCCTC ATGGCTGACA GCCCAGGTGG CTACAAAGAA | |
| TGTGGCACCA ATGAAGGCCC CCAAGAGGAT GAGAATGGCA GCAGTGCCAC TGGCAGCAGC | |
| AAGGTGCGAC AGCTCTGGTC AGAAGACTCC TGCGAATCTG GCAAGTGGAG CAGGGGCCTC | |
| CATGACCTGG GCCAGGTCTT CCAGCCTAGG CAGAACAGCA AGATGGGCGG GTGTTTCAGT | |
| CTGATCATCT CTTGAGCTTT TAGAAGGTGG AGGGGCTGTC GGCGGGAGGC AAAGCGGGTT | |
| AACCCTCGAG GACAGGCACC CACTTCTGCC TCTGCACTGG TGAGTGCCCT GCCCTCAGCA | |
| CACACAGAGT GGGTTCTCCA TGTCAGCCAG TCTCTGATGC CAGTTGTCCA GAATGCCAGT | |
| CTCTTCCTTT ACAACAAACA TGGTAACATC AGATGGGCAA GATCAGTGGA AGGGTCTGTT | |
| CTGAGTGGTG CCTGCCCCTG TGGACAGACC TTTAGGGATG GACAGATGAG CAGCAGCTGC | |
| AGGGCCAGCA AGAGCTAAGG AGCTGGGGAG TGAGTTAGTT GAATGACGGT AATTGCCGGG | |
| GGGGGGCGGG GGGGGGGTGG TGAGTCTCCA GGCTGCACTG TGAAGTGGGC GGGGCCCCAG | |
| CACTCGGTGT TTCCCTCTCT GGCTCCTCCT CCTCTAAGGT TTCCTGACTG ACAGCCTTCC | |
| CTTGTGACTG CTGCCGTCTT TCCCTAAGTC TGGTCCTGCT TTCAGGTTCC TCTCAGTACA | |
| GCCTCAGCCC GAGGTTCCCT TCCTCTTGCA TCCATGTGTG TGTTTCAGAC GCGGCCATCC | |
| TTCCCTACTT CCAGATCCTT GTAGGGCAGT TGGTGGAGGG TGGGAGGCAC CCCGGTGTTG | |
| CCTCCATGAA GCCCTGTGCC AGTCACTGGG CTGCAAGGCT GAGGAAATTG TGTCCGTGTC | |
| AGAAAGCTCC TCAGCTCAGA GGTGCTGGTA CCTCCTGCGT GGTAGGAAGG CAGGGGGAAG | |
| AGGCCCTGCT TCTCCTGTTC TCTTTGCCCT TATGAGACTT GAGAGTCTGT GTCATCTGTG | |
| CCTTGCATGT CTTTTTTTCA G | |

Exon

| | |
|---|---|
| ACTCCCTGCG ACAAGGACTG TGTACTGCAT GAATGAGGCT GAGATAGTTG ATGTTGCTCT | SEQ ID NO.6 |
| GGGAATCCTG ATTGAG | |

Intron

| | |
|---|---|
| GTAAAGTCAG ACAAGCCTCT TACTCATGAC CAGAAACTCT GCATGGGCAG AGGCTAGACC | SEQ ID NO.7 |
| CTTGGTCACC GTCAGTAAGA GAGACGCCCT CTGCCGTAGG CTACCTCTTC TTCCTCCTGC | |
| CACTTCCCTT GCTCTTATCT CGGCCATGCT GCCCAGGCCT CTCCCCATGT GGGTGTGCGC | |
| TCGTGGGGTG TCTTTGAGGC TAGAAGCCTG GGAGGGCATG CGTGTGTTAG GGAGGGCGTG | |
| TGTGTGTTAG GGAGAGCGTG ACTGTGCACT GGTGTGTATG TGGATGGGAC ATGAAGCGTT | |
| TACCTGTTTG TGTTCTTGGA AAGGTTCCCA GTTGGGTAGA TGACTTCTTA GCCATTGCCC | |
| CTCTCCTGAG CAGAGGATGC TGAGCTTCCT AGCTTCCTGC AGTAATAATT GCAGTGCCTC | |
| AGAGTTGCAC AGCATTCTAC AGTTTACCAG GTGCCTTGAA AGCATGCGTT ATGTCATTTT | |
| AGCCTTGCAA TAACCCTGTG GTGTGGGACT TTTCCTAGTG CTAAGGCATG GGCCCACACC | |
| CAGGTTTTGT ATCCTGGGTT CTGTCACCGG CCTTTCTAGA CATGTTTCTT CCATTTCTTT | |
| TTCCTTTCCC CTCAGCTAAT CCTCATGCCT TTGCTTGCTT CTCTCCCGAG TGGGTTCTGG | |
| TTTCTTCAAG AGCTGTGAAG AGGGGGTCAG GGGAAGGAGT GGGGTGAAGA GAGGGTGGGT | |
| GCTCAGGATA TGGGTACATT GCCTGGCCTG GTCACATTGG CTTTGTAGTT GCTTCTAGAT | |
| ACATCCTGTG TTACTGACAG ATCAGCATGT TAGGGAAATA AAACACGTAT GTTGAGCCTG | |
| CGTTTTCCCG TACTCCACAG | |

Exon

| | |
|---|---|
| AGCCGCAAAC AGGAAAAGGC CTGCGAGCAG CCGGCCCTGG CGGGGGCTGA TAACCCAGAG | SEQ ID NO.8 |
| CACTCCCCTC CCTGCTCCGT GTCGCCTCAC ACAAGTTCTG GGAGCAGCAG TGAGGAAGAG | |
| GACAGTGGGA AACAGGCACT GGCTCCAGGC CTCAGCCCTT CCCAGAGGCC GGGGGGTTCC | |
| AGCTCTGCCT GTAGCAGGAG CCCTGAGGAG GAGGAGGAAG AGGATGTGCT GAAATACGTC | |
| CGGGAGATCT TTTTCAGCTA GGGCATAAAC TGTGCACTGA ACTGTCTGCC GAGAGCAGCT | |
| GGAGGACAGC TGAGCTTCCA CTGGTGCTGC TGGGCCGCCC GCCTGTGGGA ATGGGGCTCT | |
| CTGTGCTCCT ACCTTTGTGC CTTCTTGGGC CTGGCAGATT CACCTCAGGC CAGAAGCCCC | |
| TGGACACTCC GGGCCTTGGG GCTGCCGTTC TGAGTGTGCG GAAGGCAGGA CTCAAAATGA | |
| GATCCCATTT GACTCCCTCT GTATGTACTG TGCCCTCTCC TGGCTCTTGA GGCTCTGGAG | |
| TCCCAATTGT CTGTGTTAGT CAGTGACCAG GTTCCAGGGA AAATGATGTC ATGTGGTGGT | |
| CCAACTTACT GGAACCAAAG AGACAGTACT T | |

We claim:

1. An isolated BTA nucleic acid molecule of at least 30 nucleotides which hybridizes to SEQ ID NO. 1, or the complement of SEQ ID NO. 1, under stringent hybridization conditions.

2. An isolated nucleic acid molecule which comprises:
(i) a nucleic acid sequence having substantial sequence identity or sequence similarity with a nucleic acid sequence of any one of SEQ. ID. NOs. 1 through 8;
(ii) nucleic acid sequences complementary to (i);
(iii) nucleic acid sequences differing from any of the nucleic acid sequences of (i) or (ii) in codon sequences due to the degeneracy of the genetic code; or
(iv) a fragment, or allelic or species variation of (i), (ii) or (iii).

3. A vector comprising a nucleic acid molecule of claim 2.

4. A host cell comprising a nucleic acid molecule of claim 2.

5. A method for preparing a protein comprising:
(a) transferring a vector as claimed in claim 3 into a host cell;
(b) selecting transformed host cells from untransformed host cells;
(c) culturing a selected transformed host cell under conditions which allow expression of the protein; and
(d) isolating the protein.

6. A protein prepared in accordance with the method of claim 5.

7. An antibody having specificity against an epitope of a protein as claimed in claim 6.

8. An antibody as claimed in claim 7 labeled with a detectable substance and used to detect the protein in biological samples, tissues, and cells.

9. A method for detecting a nucleic acid molecule as claimed in claim 2 in a biological sample comprising the steps of:
(a) hybridizing a nucleic acid molecule of claim 2 to nucleic acids of the biological sample, thereby forming a hybridization complex; and
(b) detecting the hybridization complex wherein the presence of the hybridization complex correlates with the presence of a nucleic acid molecule encoding the protein in the biological sample.

10. A method as claimed in claim 9 wherein nucleic acids of the biological sample are amplified by the polymerase chain reaction prior to the hybridizing step.

11. A method of diagnosing and monitoring cancer mediated by a tumor associated protein by determining the presence of a nucleic acid molecule encoding the tumor associated protein or the presence of the tumor associated protein wherein the tumor associated protein is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by the nucleic acid molecule as claimed in claim 2.

12. A method as claimed in claim 11 wherein the condition is breast cancer.

13. A method for preventing or treating a condition mediated by a tumor associated protein which is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by the nucleic acid molecule as claimed in claim 2, comprising administering an effective amount of an antibody specific for the tumor associated protein.

14. A method as claimed in claim 13 wherein the cancer is breast cancer.

15. A vaccine to prevent cancer and/or to treat cancer comprising a tumor associated protein which is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by the nucleic acid molecule as claimed in claim 2, or peptides derived from the protein, or synthetic peptides thereof, or any combination of these molecules.

16. A method to prevent cancer or to treat cancer in subjects who have a tumor associated protein on their cells comprising administering a vaccine for stimulating or enhancing in the subjects antibodies directed against the tumor associated protein wherein the tumor associated protein is protein phosphatase 4 regulatory subunit (Genbank AJ271448); a protein product corresponding to Genbank AK001674; putative translation initiation factor (SUI1) (Genbank NM-005801); RNA helicase (Genbank AL359945); MIL1 protein, nuclear gene encoding mitochondrial protein (NM-015367); MacMarks protein (Genbank X70326); chromosome 11 open reading frame 10 (c11orf10) (Genbank AF0867763); plakophilin 4 (PKP4) (Genbank NM-003628), or a protein encoded by the nucleic acid molecule as claimed in claim 2.

17. A method for preventing patients from having tumors prior to their occurrence comprising administering a vaccine as claimed in claim 15.

18. A method for stimulating or enhancing in a subject production of antibodies directed against a tumor associated protein comprising administering to the subject a vaccine as claimed in claim 15 in a dose effective for stimulating or enhancing production of the antibodies.

19. A methods for treating, preventing, or delaying recurrence of cancer comprising administering to the subject a vaccine as claimed in claim 15 in a dose effective for treating, preventing, or delaying recurrence of cancer.

20. Use of a vaccine as claimed in claim 15 to treat, prevent, or delay recurrence of cancer.


SEQUENCE LISTING (8 sequences)

SEQ ID NO: 1 - DNA, 1383 bp, Homo sapiens
  misc_feature (1165)..(1165): n is unknown
gcggccgctccagtcggaaggagccgttgcagccctgcccggcgtgtggcggggtcagcgaggcccctgcggtgcttcgcccgcgccttccgctccgcactcacccgccctctgggcctcccacaggtcggcgccgccagcggcgggtaccggcccgcagagggagggcgggctgtgaactggcagcacatcgcgttcccgcggggcccccattctgcccaggcagggagcggctgggatgggactgaggtcggtccgcgagcgcggtgccccagagacccggcttctggaagttgccgcggcctgctcttggggcccagaagtagggcggggaggcgcggggtctggagtctgcaggacctggctgcgaatccaaccagctgatttcaaacttttctgtgatcttctgcaagttaactaagtctcagttcctcgtttgcgaaatgtacttaataggcaccccagggtgaactaaatttgtccgcacagactgcagcattgcgcttgggaggcgccttatggggtaggagtcactaatatcactgagttttgacagaaatgaagtgcattggcaaaggaatcaaagatcaactcccactgaggacaaatggacctgtaattccgggtgtgacgagagaacgagatttaccttcctgaattaaaaaacagactccctgcgacaaggactgtgtactgcatgaatgaggctgagatagttgatgttgctctgggaatcctgattgagagccgcaaacaggaaaaggcctgcgagcagccggccctggcgggggctgataacccagagcactcccctccctgctccgtgtcgcctcacacaagttctgggagcagcagtgaggaagaggacagtgggaaacaggcactggctccaggcctcagcccttcccagaggccggggggttccagctctgcctgtagcaggagccctgaggaggaggaggaagaggatgtgctgaaatacgtccgggagatcttttgtcagctagggcataaactgtgcactgaactgtctgccgagagcagctggaggacagctgagcttccactggtgctgctgggccgcccgcctgtgggaatggggctctctgtgctcctacctttgtgccttcttgggcctggcagattcacctcaggccagaagccntcggacactccgggccttggggtgcccgttctgagtgtgcggaaggcaggactcaaaatgagatcccatttgactccctctgtatgtactgtgccctctcctggctcttgaggctctggaagtcccaattgtctgtgttagtcaagtgaccaaggttccagggaaaatgatgtcatgttggtggtccaacttacttggaaacnaaagagacagtactt

SEQ ID NO: 2 - DNA, 581 bp, Homo sapiens
cgcgcggccgctccagtcggaaggagccgttgcagccctgcccggcgtgtggcggggtcagcgaggcccctgcggtgcttcgcccgcgccttccgctccgcactcacccgccctctgggcctcccacaggtcggcgccgccagcggcgggtaccggcccgcagagggagggcgggctgtgaactggcagcacatcgcgttcccgcggggcccccattctgcccaggcagggagcggctgggatgggactgaggtcggtccgcgagcgcggtgccccagagacccggcttctggaagttgccgcggcctgctcttggggcccagaagtagggcggggaggcgcggggtctggagtctgcaggacctggctgcgaatccaaccagctgatttcaaacttttctgtgatcttctgcaagttaactaagtctcagttcctcgtttgcgaaatgtacttaataggcaccccagggtgaactaaatttgtccgcacagactgcagcattgcgcttgggaggcgccttagggggtaggagtcactaatatcactgagttttgacagaaatgaaggtgcattggcaaag

SEQ ID NO: 3 - DNA, 886 bp, Homo sapiens
gtgtgccccatggccgggcaggttaggaggagggcctggtaatattttttcttaaattgtaaacagccatctggatgagcaatgcattatcaaattatgattcagagaggactatcgctgactactcttttttttgggcaaacctccgcttcaggagcttctggcttgattcctaagtggaaggtagagacctaggccctctgagtcacaactccatctctctgggtgaggactgagctccaggactgctgagtggaggcagaacaattgggatagggaaaagagaggccaaactagatcagaggctggcgtgggcttcagaatctacagactggcacagttaatgcctccgggcccctattgctgcttcaagtttgaccaatcagaagtatctttagcttaagggcggcgtgttggccaatgagactttattgtgaaataaaatgccctcagtttcatttaactgagccaccatagaagagtagagaaatcgagttggccagatgagggagaggccattaagaggtattttagggattgaatgggctgggcacccagaggacagtggcatttggggtggtaatgagaaggaacattaggggcctccgcctccttccgttcctcctgtgctgagtcagtcagcacagaggctgcaggaggtatctctggttggctttggctgcctgtagggggcaccagctttggggaggtcagagggctctctcctgagctgctgtcctgcccacaatcacacctgcctgatgcctgtgaatggccgtctgagtctcttgggccttggtcacttctggcctgccctgcaccgaccagtaactgtgcctgatgactggaggtatgggaattcaccggactttattgttctttgtag

SEQ ID NO: 4 - DNA, 92 bp, Homo sapiens
gaatcaaagatcaactcccactgaggacaaatggacctgtaattccgggtgtgacgagagaacgagatttaccttcctgaattaaaaaacag

SEQ ID NO: 5 - DNA, 1161 bp, Homo sapiens
gtcattaagcttgggccctgactcttctttgtgagaaggtacagagatggaaaccttacaatccgagactaaaacgagggtccttccctcatggctgacagcccaggtggctacaaagaatgtggcaccaatgaaggcccccaagaggatgagaatggcagcagtgccagtggcagcagcaaggtgcgacagctctggtcagaagactcctgcgaatctggcaagtggagcaggggcctccatgacctgggccaggtcttccagcctaggcagaacagcaagatgggcgggtgtttcagtctgatcatctcttgagcttttagaaggtggaggggctgtgggcgggaggcaaagcgggttaaccctcgaggacaggcacccacttctgcctctgcactggtgagtgccctgccctcagcacacacagagtgggttctccatgtcagccagtctctgatgccagttgtccagaatgccagtctcttcctttacaacaaacatggtaacatcagatgggcaagatcagtggaagggtctgttctgagtggtgcctgcccctgtggacagacctttagggatggacagatgagcagcagctgcagggccagcaagagctaaggagctggggagtgagttagttgaatgacggtaattgccgggggggggcggggggggggtggtgagtctccaggctgcactgtgaagtgggcggggccccagcactcggtgtttccctctctggctcctcctcctctaaggtttcctgactgacagccttcccttgtgactgctgccgtctttccctaagtctggtcctgctttcaggttcctctcagtacagcctcagcccgaggttcccttcctcttgcatccatgtgtgtgtttcagaggcggccatccttccctacttccagatccttgtagggcagttggtggagggtgggaggcaccccggtgttgcctccatgaagccctgtgccagtcactgggctgcaaggctgaggaaattgtgtccgtgtcagaaagctcctcagctcagaggtgctggtacctcctgcgtggtaggaaggcagggggaagaggccctgcttctcctgttctctttgcccttatgagacttgagagtctgtgtcatctgtgccttgcatgtctttttttcag

SEQ ID NO: 6 - DNA, 76 bp, Homo sapiens
actccctgcgacaaggactgtgtactgcatgaatgaggctgagatagttgatgttgctctgggaatcctgattgag

SEQ ID NO: 7 - DNA, 860 bp, Homo sapiens
gtaaagtcagacaagcctcttactcatgaccagaaactctgcatgggcagaggctagacccttggtcaccgtcagtaagagagacgccctctgccgtaggctacctcttcttcctcctgccacttcccttgctcttatctcggccatgctgcccaggcctctccccatgtgggtgtgcgctcgtggggtgtctttgaggctagaagcctgggagggcatgcgtgtgttagggagggcgtgtgtgtgttagggagagcgtgactgtgcactggtgtgtatgtggatgggacatgaagcgtttacctgtttgtgttcttggaaaggttcccagttgggtagatgacttcttagccattgcccctctcctgagcagaggatgctgagcttcctagcttcctgcagtaataattgcagtgcctcagagttgcacagcattctacagtttaccaggtgccttgaaagcatgcgttatgtcattttagccttgcaataaccctgtggtgtgggacttttcctagtgctaaggcatgggcccacacccaggttttgtatcctgggttctgtcaccggcctttctagacatgtttcttccatttctttttcctttcccctcagctaatcctcatgcctttgcttgcttctctcccgagtgggttctggtttcttcaagagctgtgaagagggggtcaggggaaggagtggggtgaagagagggtgggtgctcaggatatgggtacattgcctggcctggtcacattggctttgtagttgcttctagatacatcctgtgttactgacagatcagcatgttagggaaataaaacacgtatgttgagcctgcgttttcccgtactccacag

SEQ ID NO: 8 - DNA, 631 bp, Homo sapiens
agccgcaaacaggaaaaggcctgcgagcagccggccctggcgggggctgataacccagagcactcccctccctgctccgtgtcgcctcacacaagttctgggagcagcagtgaggaagaggacagtgggaaacaggcactggctccaggcctcagcccttcccagaggccggggggttccagctctgcctgtagcaggagccctgaggaggaggaggaagaggatgtgctgaaatacgtccgggagatctttttcagctagggcataaactgtgcactgaactgtctgccgagagcagctggaggacagctgagcttccactggtgctgctgggccgcccgcctgtgggaatggggctctctgtgctcctacctttgtgccttcttgggcctggcagattcacctcaggccagaagcccctggacactccgggccttggggctgccgttctgagtgtgcggaaggcaggactcaaaatgagatcccatttgactccctctgtatgtactgtgccctctcctggctcttgaggctctggagtcccaattgtctgtgttagtcagtgaccaggttccagggaaaatgatgtcatgtggtggtccaacttactggaaccaaagagacagtactt